SEQUENCE LISTING (25 sequences)

SEQ ID NO: 1 - DNA, 1821 bp, Artificial Sequence
  Description of Artificial Sequence Synthetic polynucleotide
  Genex1_Hans_sec_final_ecor1.dna
gaattcatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcgccccagcttctgctgttccagctaccattccattgaccatcaccaacgactctggcagaggtccaatctacctgtacgttttgggtgagagagatggcgttgctggttgggctgatgctggtggtacttttcatccatggccaggtggtgtgggtccagttccagttcctgctccagacgcatctattgctggtccaggtccaggacagtctgttaccattagactgccaaagctgtcgggcagagtgtactactcttacggtcagaagatgaccttccagatcgtgctggacggaagattggttcagccagctgttcagaacgactcggacccaaacagaaacatcctgttcaactggaccgagtacaccttgaacgatggtggtctgtggatcaactctacccaagttgaccattggtctgccccataccaggttggtgttcaacgcgctgatggtcaggttttgtctaccggtatgctgaagccaaacggttacgaggctttctacaccgctttggaatctgctggatggggtggtttggttcaaagagcaccagacggttctagactgagagcattgaacccatctcacggtatcgacgtgggcaagatttcttctgcctcgatcgactcttacgtgaccgaggtttggaactcgtacagaaccagagacatgtgcgtgaccccattttctcacgagccaggtactcagttcagaggtagagtggatggtgactggttcagattcagaaacggctctggtcaagaggttgccgcttttaagaaaccagacgcctcttctgtgtacggctgtcacaaagatcttcaggctccaaacgaccacgttgtgggaccaattgctagaaccttgtgtgccgctctggttagaaccactgctctgactaacccaaaccagccagatgctaactctgccggtttttaccaggacgccagaactaacgtttacgccaaattggctcaccagcagatggccaacggtaaggcttacgcttttgctttcgacgacgttggtgctcacgagtctttggttcatgacggtaatccacaggccgcctacattaagttggacccattcactggtactgctaccccaattgcaaacggtggttctactgagcaacctggaactccaggtggtttgccagctggtactggtgctttgagaattggttctaccctgtgtctggacgttccatgggctgatccaactgacactaaccaggttcagctggctacctgttctggtaacgctgctcaacaatggaccagaggtactgacggtactgttagagccctgggaaagtgtttggacgtggctagatctggaactgctgatggtacagccgtgtggatctacacctgtaatggaactggtgctcagaagtggacctacgactctgctactaaggctctgagaaacccacagtctggtaagtgcttggatgctcaaggtggtgctccattgagagatggtcagaaagttcagctgtggacctgcaaccagactgaggctcaaagatggaccctgtaaggatcc

SEQ ID NO: 2 - DNA, 1917 bp, Artificial Sequence
  Description of Artificial Sequence Synthetic polynucleotide
  H1_Hans_artes_sec_ecor1_final.dna
gaattcatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcgacaccctgtgtattggttaccacgccaacaactctaccgacaccgttgacactgtgttggagaagaacgtgaccgtgactcactctgtgaacctgttggaggacaagcacaacggtaagctgtgtaagctgagaggtgttgctccactgcacctgggcaaatgtaatatcgctggttggattctgggtaacccagagtgtgagtctttgtctaccgcctcgtcttggtcgtacatcgttgagactccatcgtctgacaacggcacttgttaccctggtgacttcatcgactacgaggaactgagagaacagctgtcgtctgtgtcgtcgttcgagagattcgagatcttcccaaagacctcgtcgtggccaaaccacgactctaacaaaggtgttaccgctgcttgtccacacgctggtgctaagtctttctacaagaacctgatctggctggtgaagaagggcaactcttacccaaagctgtcgaagtcctacatcaacgacaagggcaaagaggtgctggttctttggggtatccaccatccatctacctctgctgaccagcagtctctttaccagaacgctgacacctacgtgttcgtgggttcgtcgagatactcgaagaagttcaagccagagatcgccatcagaccgaaggtgagagatcaagagggcagaatgaactactactggaccttggttgagccaggcgacaagattacctttgaggctaccggtaacctggtggttccaagatacgctttcgccatggaaagaaacgccggctctggtatcatcatctctgacactccagtgcacgactgtaacaccacttgtcagactccaaagggtgccattaacacctcgctgccattccagaacattcacccaatcaccatcggcaagtgccctaagtacgtgaagtctaccaagctgagactggctaccggactgagaaacatcccatcgattcagtctagaggcctgttcggtgctatcgccggttttattgaaggtggttggaccggtatggtggacggttggtatggttaccatcaccagaacgagcaaggttctggttacgctgctgacctgaaatctacccagaacgccattgacgagatcaccaacaaggtgaactcggtgatcgagaagatgaacacccagttcaccgctgtgggtaaagagttcaaccacctcgagaagagaatcgagaacctgaacaagaaggtggacgacggtttcctggacatctggacttacaacgctgagctgctggtgttgcttgagaacgagagaactttggactaccacgattcgaacgtgaagaacctctacgagaaggttagatcgcagctgaagaacaacgctaaagagatcggcaacggctgcttcgagttctaccacaagtgtgacaacacgtgcatggaatcggtgaagaacggaacctacgactacccaaagtactcggaggaagccaagctgaacagagaagagattgacggcgtgaagttggagtcgaccagaatctaccagatcctggccatctactctaccgtggcttcttctctggttctggtggtttctctgggtgccatctctttctggatgtgctctaacggttctctgcagtgcagaatctgcatctaaggatcc

SEQ ID NO: 3 - DNA, 771 bp, Artificial Sequence
  Description of Artificial Sequence Synthetic polynucleotide
  M1_Hans_mun1_final.dna
caattgatgtctctgctgaccgaggttgagacttacgtgctgtctatcatcccatctggtccactgaaggctgagattgctcagagattggagtctgtgttcgccggtaagaacactgacttggaggctctgatggaatggctgaaaaccagaccaatcctgtcgccactgaccaagggtattctgggtttcgttttcaccctgaccgtgccatctgagagaggattgcaaagaagaagattcgtccagaacgccctgaacggtaacggtgatccaaacaacatggacagagccgtgaagctgtacaagaagctgaagagagagatcaccttccacggcgccaaagaggtttctctgtcttactctactggtgccctggcctcttgtatgggtctgatctacaacagaatgggcaccgttactaccgaggctgcttttggtttggtttgtgctacctgtgagcagatcgctgactctcaacacagatcgcacagacagatggctaccacaactaacccactgatcagacacgagaaccgcatggttttggcttctaccactgccaaggctatggaacagatggcaggttcttctgagcaagctgctgaggcaatggaagttgccaaccagactagacaaatggtgcacgccatgagaaccatcggtactcatccatcttcgtctgctggtctgaaggacgacttgttggagaacttgcaggcctaccagaaaagaatgggtgtgcagatgcagagattcaagtaaagatct

SEQ ID NO: 4 - DNA, 1476 bp, Artificial Sequence
  Description of Artificial Sequence Synthetic polynucleotide
  SecChitinase_hans_final_mun1.dna
caattgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcaccccagtgtctaccaacgacgtgtcggttgagaaaagagcctctggttacaccaacgccgtgtactttacaaactggggcatctacggcagaaacttccagccacaagacttggttgcctctgacatcacccacgtgatctacccattcatgaacttccaggctgacggaaccgttgtttctggtgatgcttacgctgactaccagaagcactactcggacgactcttggaacgacgttggtaacaacgcctacggttgtgtgaaccagctgttcaagctgaagaaggccaacagaaacctgaaggtgatgctgtctattggcggttggacttggtcgactaacttcccatctgctgcttctaccgacgctaacagaaagaacttcgccaagaccgccatcacgttcatgaaggattggggtttcgacggtatcgacgttgactgggaatacccagctgacgatactcaggctaccaacatggttctgctgctgaaagagatcagatcgcagttggacgcttacgcagctcaatacgctccaggttaccacttcctgttgtctattgctgctcctgctggtccagagcattactctgcattgcacatggctgaccttggtcaggttctggactacgttaacctgatggcttacgactacgctggctcttggtcgtcttactctggtcacgatgctaacctgttcgctaacccatcgaacccaaactcgtcgccatacaacactgaccaggccatcaaggcctacatcaacggtggtgttccagcctctaagatcgttctgggtatgccaatctacggtagatcgttcgagtccaccaacggtattggtcagacctacaacggtatcggctctggttcttgggagaacggtatttgggactacaaggtgttgccaaaggctggtgctactgtgcagtacgactctgttgctcaggcctactactcttacgactcgtcgtctaaagagctgatctcgtttgacaccccagacatggtgtcgaagaaggtgtcttacctgaagaaccttggccttggcggttcgatgttttgggaagcttctgctgacaagaccggctctgactctctgattggtacttctcacagagccctgggttctttggactctactcagaacttgctgtcgtaccctaacagccagtacgacaacatcagatcgggcctgaactaaagatct

SEQ ID NO: 5 - DNA, 534 bp, Artificial Sequence
  Description of Artificial Sequence Synthetic polynucleotide
  Genex3_Hans_sec_final_ecor1.dna
gaattcatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcggtgacggttacctgatcatgtgcaagaactgtgacccaaacaccggttcttgtgactggaagcagaactggaacacctgtgttggtatcggtgctaacgtgcactggatggttactggtggttctactgacggtaagcagggttgtgctaccatctgggaaggttctggttgtgtgggaagatctaccaccatgtgctgtccagctaacacttgctgcaacatcaacaccggcttctacatcagatcgtacagaagagtcgagtaaggatcc

SEQ ID NO: 6 - DNA, 1935 bp, Artificial Sequence
  Description of Artificial Sequence Synthetic polynucleotide
  GAG MS2 Fusion Protein
atgggtgctagagcttctgttttgtctggtggtgaattggatagatgggaaaagatcagattaagaccaggtggtaagaagaagtacaagttgaaacatatcgtttgggcctccagagaattggaaagatttgctgttaatccaggcttgttggaaacttctgaaggttgcagacaaatcttgggtcaattgcaaccatctttacaaaccggttctgaagaactgagatccttgtataatactgttgctaccttgtactgcgtccatcaaagaattgaaatcaaggataccaaagaggccttggacaagattgaagaagaacagaacaagtccaagaagaaggctcaacaagctgctgctgatactggtcattctaatcaagtttctcagaactacccaatcgtccaaaacattcaaggtcaaatggttcatcaagccatttctccaagaactttgaatgcatgggtcaaagttgtagaagaaaaggctttttcacctgaggttatcccaatgttttctgctttatctgaaggtgctactccacaagatttgaacactatgttgaacactgttggtggtcatcaagctgctatgcaaatgttgaaagaaaccattaacgaagaggctgctgaatgggatagagttcatccagttcatgctggtccaattgcacctggtcaaatgagagaacctagaggttctgatattgctggtactacttctaccttgcaagaacaaattggttggatgactcataacccaccaattccagttggtgaaatctacaagagatggatcattttgggcttgaacaagatcgtcagaatgtactctccaacctccattttggatattagacagggtccaaaagaaccattcagagattacgttgacagattctacaagaccttgagagctgaacaagcttctcaagaggttaagaattggatgaccgaaaccttgttggttcaaaatgctaatccagattgcaagaccatcttgaaagctttaggtccaggtgctacattggaagaaatgatgactgcttgtcaaggtgtaggtggtccaggtcataaggctagagttttggctgaagctatgtctcaagttactaatccagccaccattatgatccaaaagggcaactttagaaaccagagaaagaccgttaagtgcttcaactgtggtaaagaaggtcatattgctaagaactgtagagccccaagaaaaaaaggttgttggaagtgcggaaaagagggtcatcaaatgaaggattgcactgaaagacaagctaacttcttgggtaaaatttggccatctcataagggtagaccaggtaactttttacaatctagaccagaacctactgctccaccagaagaatcttttagatttggtgaagaaaccaccactccatctcaaaagcaagaacctatcgacaaagagttgtatccattggcttcattgagatctttgttcggttctgatccatcatctcaaggtggttctacatcttctgaattctgctctagaagatatagaggtcctggtattcatagaccagttgctactatggcttctaacttcactcaattcgttttggttgataacggtggtactggtgatgttacagttgctccatctaatttcgctaacggtattgcagaatggatctcctctaattctagatcccaagcttacaaggttacctgctctgttagacaatcttctgctcagaatagaaagtacaccatcaaggttgaagttccaaaaggtgcttggagatcttacttgaacatggaattgaccattccaattttcgccaccaactctgattgtgaattgatcgttaaggctatgcagggtttgttgaaggatggtaatccaattccatctgctattgctgctaattctggtatctatgctgctgcttaa

SEQ ID NO: 7 - DNA, 1394 bp, Artificial Sequence
  Description of Artificial Sequence Synthetic polynucleotide
  yGFPMS2_sal_not
gtcgacatggtgtctaaaggtgaagagttgttcactggtgttgttccaattttggttgaattggatggtgatgttaacggccataagttttctgtttctggtgaaggtgagggtgatgctacttatggtaaattgactttgaagttcatctgcaccacaggtaaattgccagttccatggccaactttggttactactttgacttatggtgtccagtgcttttctagatacccagatcatatgaagcaacacgactttttcaaatccgctatgccagaaggttacgttcaagaaagaaccatcttcttcaaggatgacggtaactacaaaactagagccgaagttaagttcgaaggtgataccttggttaacaggattgaattgaagggcatcgattttaaagaggacggtaacattttgggtcacaagttggagtacaactacaactctcataacgtttacatcatggccgacaagcaaaagaacggtattaaggttaacttcaagatcaggcacaacatcgaagatggtagtgttcaattggctgatcactaccaacaaaacactccaattggtgatggtccagttttgttgccagataaccattacttgtctacccaatctgctttgtctaaggacccaaacgaaaaaagggatcacatggtcttgttggaatttgttactgctgctggtattaccttaggtatggacgaattatacaagtaaggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatctgcggccgc

SEQ ID NO: 8 - DNA, 19 bp, Artificial Sequence
  Description of Artificial Sequence Synthetic oligonucleotide
  Synonymous MS2
  modified_base (1)..(1): a, c, t, g, unknown or other
  modified_base (3)..(3): a, c, t, g, unknown or other
  modified_base (10)..(10): a, c, t, g, unknown or other
  modified_base (16)..(17): a, c, t, g, unknown or other
  modified_base (19)..(19): a, c, t, g, unknown or other
nrndsassancasssnnyn

SEQ ID NO: 9 - DNA, 4011 bp, Artificial Sequence
  Description of Artificial Sequence Synthetic polynucleotide
  Synonymous MS2 24x array
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatcccaataccggtaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgagcatacattgtgcctatttcttgggtggacgatcacgccacccatgctctcacgaatttcaaaacacggacaaggacgagcaccaccagggctcgtcgttccacgtccaatacgattacttacctttcgggatcacgatcacggatcccgcagctacatcacttccactcaggacattcaagcatgcacgatcacggcatgctccacaagtctcaaccacagaaactaccaaatgggttcagcaccagcgaacccactcctacctcaaacctcttcccacaaaactggcaagcaggatcaccgcttgcccattccaacataccaaatcaaaaacaattactggtacagcatcagcgtaccagcccacatctctcactactatcaaaaaccaaaccgttcagcaacagcgaacggtacacacggaaaaatcaactggtttacaaatacgaaagacgatcacgctttcgtccagcgcaaactattacgaaaaacatccgacgggaagagcaacagccttcccgcggcggaaaacctcacaaaaacacgacaaacggatgcacgaacacggcatccgccgacaacccacaaacttacaaccaggcaaacggtgcaggatcaccgcaccgtacatcaaacacctcagatctcatatcgatattgggtcatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - protein, 548 aa, Artificial Sequence
  Description of Artificial Sequence Synthetic polypeptide
  1,3 Betaglucanase
MPHDRKNSSRRAWAALCAAVLAVSGALVGVAAPASAVPATIPLTITNDSGRGPIYLYVLGERDGVAGWADAGGTFHPWPGGVGPVPVPAPDASIAGPGPGQSVTIRLPKLSGRVYYSYGQKMTFQIVLDGRLVQPAVQNDSDPNRNILFNWTEYTLNDGGLWINSTQVDHWSAPYQVGVQRADGQVLSTGMLKPNGYEAFYTALESAGWGGLVQRAPDGSRLRALNPSHGIDVGKISSASIDSYVTEVWNSYRTRDMCVTPFSHEPGTQFRGRVDGDWFRFRNGSGQEVAAFKKPDASSVYGCHKDLQAPNDHVVGPIARTLCAALVRTTALTNPNQPDANSAGFYQDARTNVYAKLAHQQMANGKAYAFAFDDVGAHESLVHDGNPQAAYIKLDPFTGTATPIANGGSTEQPGTPGGLPAGTGALRIGSTLCLDVPWADPTDTNQVQLATCSGNAAQQWTRGTDGTVRALGKCLDVARSGTADGTAVWIYTCNGTGAQKWTYDSATKALRNPQSGKCLDAQGGAPLRDGQKVQLWTCNQTEAQRWTL

SEQ ID NO: 11 - protein, 337 aa, Artificial Sequence
  Description of Artificial Sequence Synthetic polypeptide
  Beta-mannanase
AHTVSPVNPNAQQTTKTVMNWLAHLPNRTENRVLSGAFGGYSHDTFSMAEADRIRSATGQSPAIYGCDYARGWLETANIEDSIDVSCNGDLMSYWKNGGIPQISLHLANPAFQSGHFKTPITNDQYKKILDSSTAEGKRLNAMLSKIADGLQELENQGVPVLFRPLHEMNGEWFWWGLTSYNQKDNERISLYKQLYKKIYHYMTDTRGLDHLIWVYSPDANRDFKTDFYPGASYVDIVGLDAYFQDAYSINGYDQLTALNKPFAFTEVGPQTANGSFDYSLFINAIKQKYPKTIYFLAWNDEWSPAVNKGASALYHDSWTLNKGEIWNGDSLTPIVE

SEQ ID NO: 12 - protein, 487 aa, Artificial Sequence
  Description of Artificial Sequence Synthetic polypeptide
  Chitinase
MRFPSIFTAVLFAASSALAAPVNTTTEDETAQIPAEAVIGYSDLEGDFDVAVLPFSNSTNNGLLFINTTIASIAAKEEGVSLEKRTPVSTNDVSVEKRASGYTNAVYFTNWGIYGRNFQPQDLVASDITHVIYPFMNFQADGTVVSGDAYADYQKHYSDDSWNDVGNNAYGCVNQLFKLKKANRNLKVMLSIGGWTWSTNFPSAASTDANRKNFAKTAITFMKDWGFDGIDVDWEYPADDTQATNMVLLLKEIRSQLDAYAAQYAPGYHFLLSIAAPAGPEHYSALHMADLGQVLDYVNLMAYDYAGSWSSYSGHDANLFANPSNPNSSPYNTDQAIKAYINGGVPASKIVLGMPIYGRSFESTNGIGQTYNGIGSGSWENGIWDYKVLPKAGATVQYDSVAQAYYSYDSSSKELISFDTPDMVSKKVSYLKNLGLGGSMFWEASADKTGSDSLIGTSHRALGSLDSTQNLLSYPNSQYDNIRSGLN

SEQ ID NO: 13 - protein, 644 aa, Artificial Sequence
  Description of Artificial Sequence Synthetic polypeptide
  GAG-MS2 Fusion
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQGGSTSSEFCSRRYRGPGIHRPVATMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAAA

SEQ ID NO: 14 - protein, 763 aa, Artificial Sequence
  Description of Artificial Sequence Synthetic polypeptide
  GAG-GFP Fusion
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQGGSTSSEFCSRRYRGPGIHRPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 15 - protein, 566 aa, Artificial Sequence
  Description of Artificial Sequence Synthetic polypeptide
  H1 Protein
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 16 - protein, 252 aa, Artificial Sequence
  Description of Artificial Sequence Synthetic polypeptide
  Influenza Matrix
MSLLTEVETYVLSIIPSGPLKAEIAQRLESVFAGKNTDLEALMEWLKTRPILSPLTKGILGFVFTLTVPSERGLQRRRFVQNALNGNGDPNNMDRAVKLYKKLKREITFHGAKEVSLSYSTGALASCMGLIYNRMGTVTTEAAFGLVCATCEQIADSQHRSHRQMATTTNPLIRHENRMVLASTTAKAMEQMAGSSEQAAEAMEVANQTRQMVHAMRTIGTHPSSSAGLKDDLLENLQAYQKRMGVQMQRFK

SEQ ID NO: 17 - protein, 469 aa, Artificial Sequence
  Description of Artificial Sequence Synthetic polypeptide
  N1 Protein
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 18 - protein, 551 aa, Artificial Sequence
  Description of Artificial Sequence Synthetic polypeptide
  PIV5 fusion glycoprotein
MGIIIQFLVVSCLLAGAGGLDPAALMQIGVIPTNVRQLMYYTEASSAFIVVKLMPTIDSPISGCNITSISSYNATVTKLLQPIGENLETIRNQLIPTRRRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFFTRFVLFDGIVYANCRSMLCKCMQPAAVILQPSSSPVTVIDMYKCVSLQLDNLRFTITQLANVTYNSTIKLESSQILPIDPLDISQNLAAVNKSLSDALQHLAQSDTYLSAITSATTTSVLSIIAICLGSLGFILIILLSVVVWKLLTIVAANRNRMENFVYHNQAFHHSQSDLSEKNQPATLGTR

SEQ ID NO: 19 - protein, 2255 aa, Artificial Sequence
  Description of Artificial Sequence Synthetic polypeptide
  PIV5 L protein
MAGSREILLPEVHLNSPIVKHKLYYYILLGNLPNEIDLDDLGPLHNQNWNQIAHEESNLAQRLVNVRNFLITHIPDLRKGHWQEYVNVILWPRILPLIPDFKINDQLPLLKNWDKLVKESCSVINAGTSQCIQNLSYGLTGRGNLFTRSRELSGDRRDIDLKTVVAAWHDSDWKRISDFWIMIKFQMRQLIVRQTDHNDPDLITYIENREGIIIITPELVALFNTENHTLTYMTFEIVLMISDMYEGRHNILSLCTVSTYLNPLKKRITYLLSLVDNLAFQIGDAVYNIIALLESFVYAQLQMSDPIPELRGQFHAFVCSEILDALRGTNSFTQDELRTVTTNLISPFQDLTPDLTAELLCIMRLWGHPMLTASQAAGKVRESMCAGKVLDFSTIMKTLAFFHTILINGYRRKHHGVWPPLNLPGNASKGLTELMNDNTEISYEFTLKHWKEISLIKFKKCFDADAGEELSIFMKDKAISAPKQDWMSVFRRSLIKQRHQHHQVPLPNPFNRRLLLNFLEDDKFDPNVELQYVTSGEYLHDDTFCASYSLKEKEIKPDGRIFAKLTKRMRSCQVIAESLLANHAGKLMKENGVVMNQLSLTKSLLTMSQIGIISEKARKSTRDNINQPGFQNIQRNKSHHSKQVNQRDPSDDFELAASFLTTDLKKYCLQWRYQTIIPFAQSLNRMYGYPHLFEWIHLRLMRSTLYVGDPFNPPADTSQFDLDKVINGDIFIVSPRGGIEGLCQKAWTMISISVIILSATESGTRVMSMVQGDNQAIAVTTRVPRSLPTLEKKTIAFRSCNLFFERLKCNNFGLGHHLKEQETIISSHFFVYSKRIFYQGRILTQALKNASKLCLTADVLGECTQSSCSNLATTVMRLTENGVEKDICFYLNIYMTIKQLSYDIIFPQVSIPGDQITLEYINNPHLVSRLALLPSQLGGLNYLSCSRLFNRNIGDPVVSAVADLKRLIKSGCMDYWILYNLLGRKPGNGSWATLAADPYSINIEYQYPPTTALKRHTQQALMELSTNPMLRGIFSDNAQAEENNLARFLLDREVVFPRVAHIIIEQTSVGRRKQIQGYLDSTRSIMRKSLESKPLSNRKLNEILDYNINYLAYNLALLKNAIEPPTYLKAMTLETCSIDIARSLRKLSWAPLLGGRNLEGLETPDPIEITAGALIVGSSYCEQCAAGDNRFTWFFLPSGIEIGGDPRDNPPIRVPYIGSRTDERRVASMAYIRGASSSLKAVLRLAGVYIWAFGDTLENWIDALDLSHTRVNITLEQLQSLTPLPTSANLTHRLDDGTTTLKFTPASSYTFSSFTHISNDEQYLTINDKTADSNIIYQQLMITGLGILETWNNPPINRTFEESTLHLHTGASCCVRPVDSCIISEALTVKPHITVPYSNKFVFDEDPLSEYETAKLESLSFQAQLGNIDAVDMTGKLTLLSQFTARQIINAITGLDESVSLTNDAIVASDYVSNWISECMYTKLDELFMYCGWELLLELSYQMYYLRVVGWSNIVDYSYMILRRIPGAALNNLASTLSHPKLFRRAINLDIVAPLNAPHFASLDYIKMSVDAILWGCKRVINVLSNGGDLELVVTSEDSLILSDRSMNLIARKLTLLSLIHHNGLELPKIKGFSPDEKCFALTEFLRKVVNSGLSSIENLSNFMYNVENPRLAAFASNNYYLTRKLLNSIRDTESGQVAVTSYYESLEYIDSLKLTPHVPGTSCIEDDSLCINDYIIWIIESNANLEKYPIPNSPEDDSNFHNFKLNAPSHHTLRPLGLSSTAWYKGISCCRYLERLKLPQGDHLYIAEGSGASMTIIEYLFPGRKIYYNSLFSSGDNPPQRNYAPMPTQFIESVPYKLWQAHTDQYPEIFEDFIPLWNGNAAMTDIGMTACVEFIINRVGPRTCSLVHVDLESSASLNQQCLSKPIINAIITATTVLCPHGVLILKYSWLPFTRFSTLITFLWCYFERITVLRSTYSDPANHEVYLICILANNFAFQTVSQATGMAMTLTDQGFTLISPERINQYWDGHLKQERIVAEAIDKVVLGENALFNSSDNELILKCGGTPNARNLIDIEPVATFIEFEQLICTMLTTHLKEIIDITRSGTQDYESLLLTPYNLGLLGKISTIVRLLTERILNHTIRNWLILPPSLRMIVKQDLEFGIFRITSIFNSDRFLKLSPNRKYLIAQLTAGYIRKLIEGDCNIDLTRPIQKQIWKALGCVVYCHDPMDQRESTEFIDININEEIDRGIDGEEI

SEQ ID NO: 20 - protein, 509 aa, Artificial Sequence
  Description of Artificial Sequence Synthetic polypeptide
  PIV5 NP Protein
MSSVLKAYERFTLTQELQDQSEEGTIPPTTLKPVIRVFILTSNNPELRSRLLLFCLRIVLSNGARDSHRFGALLTMFSLPSATMLNHVKLADQSPEADIERVEIDGFEEGSFRLIPNARSGMSRGEINAYAALAEDLPDTLNHATPFVDSEVEGTAWDEIETFLDMCYSVLMQAWIVTCKCMTAPDQPAASIEKRLQKYRQQGRINPRYLLQPEARRIIQNVIRKGMVVRHFLTFELQLARAQSLVSNRYYAMVGDVGKYIENCGMGGFFLTLKYALGTRWPTLALAAFSGELTKLKSLMALYQTLGEQARYLALLESPHLMDFAAANYPLLYSYAMGIGYVLDVNMRNYAFSRSYMNKTYFQLGMETARKQQGAVDMRMAEDLGLTQAERTEMANTLAKLTTANRGADTRGGVNPFSSVTGTTQVPASATGDTFEGYMAADRLRQRYADAGTYDDEMPPLEEEKEDDTSAGPRTGPTLEQVALDIQNAAVGAPIHTDDLNAALGDLDI

SEQ ID NO: 21 - protein, 392 aa, Artificial Sequence
  Description of Artificial Sequence Synthetic polypeptide
  PIV5 V/P Protein
MDPTDLSFSPDEINKLIETGLNTVEYFTSQQVTGTSSLEKNTIPPGVTGLLTNAAEAKIQESTNHQKGSVGGGAKPKKPRPKIAIVPADDKTVPGKPIPNPLLGLDSTPSTQTVLDLSGKTLPSGSYKGVKLAKFGKENLMTRFIEEPRENPIATSFPIDFKRGAGIPAGSIEGSTQLDGWEMKSRSLSGAIHPVLQSPLQQGDLNALVTSVQSLALNVNEILNTVRNLDSRMNQLETKVDRILSSQSLIQTIKNDIVGLKAGMATLEGMITTVKIMDPGVPSNVTVEDVRKTLSNHAVVVPESFNDSFLTQSEDVISLDELARPTATSVKKIVRKVPPQKDLTGLKITLEQLAKDCISKPKMREEYLLKINQASSEAQLIDLKKAIIRSAI

SEQ ID NO: 22 - protein, 419 aa, Artificial Sequence
  Description of Artificial Sequence Synthetic polypeptide
  COVID-19 S protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 23 - protein, 125 aa, Artificial Sequence
  Description of Artificial Sequence Synthetic polypeptide
  Killer Toxin
MKFSFVYGLTGFLAATSSALPSEILSTGYERSALEKRGDGYLIMCKNCDPNTGSCDWKQNWNTCVGIGANVHWMVTGGSTDGKQGCATIWEGSGCVGRSTTMCCPANTCCNINTGFYIRSYRRVE

SEQ ID NO: 24 - DNA, 1422 bp, Artificial Sequence
  Description of Artificial Sequence Synthetic polynucleotide
  Neuraminidase
caattgatgaacccgaaccagaagatcatcaccatcggctctgtgtgcatgaccattggtatggccaacctgatcctgcagatcggcaacatcatctcgatctggatctcgcactctatccagctgggtaaccagaaccagattgagacttgcaaccagtccgtgatcacctacgagaacaacacctgggtgaatcagacctacgtgaacatctcgaacaccaactttgccgccggtcagtctgtggtttctgttaagttggctggcaactcttcgctgtgtccagtttctggttgggccatctactcgaaggacaactctgtgagaatcggctctaagggtgacgtgttcgtgatcagagagccattcatctcttgctcgcctttggagtgcagaacattctttttgacccagggtgctctgctgaacgacaagcactctaacggaaccatcaaggacagatcgccatacagaaccctgatgtcttgtccaatcggtgaggtgccatctccatacaactcgagattcgagtctgttgcctggtctgcttctgcttgtcacgacggtattaactggctgaccatcggtatttctggcccagataatggtgccgttgccgtgttgaagtacaacggtattatcaccgacaccatcaagtcgtggcgcaacaacattctgagaacccaagagtctgagtgcgcctgtgttaacggttcttgtttcaccgttatgaccgacggtccatctaacggtcaggcctcttacaaaatcttcagaatcgagaagggcaagatcgtgaagtcggttgagatgaacgctccgaactaccactacgaggaatgctcttgttacccagactcgtcggagatcacctgtgtgtgtagagataactggcacggctctaacagaccatgggtgtctttcaaccagaacttggagtaccagatcggttacatctgctctggcatcttcggtgacaacccaagaccaaacgacaagactggttcttgtggtccagtgtcgtctaacggtgcaaacggtgttaagggcttctcgttcaagtacggtaacggtgtttggatcggcagaactaagtcgatctcgtcgagaaacggctttgagatgatctgggatccaaacggttggaccggtactgacaacaacttctcgatcaagcaggacatcgtgggcatcaacgaatggtctggttactctggttctttcgtgcagcacccagagttgactggtctggactgtatcagaccatgcttctgggttgagctgatcagaggcagaccaaaagagaacaccatctggacctcgggctcgtctatttctttctgcggtgttaactctgacaccgttggatggtcttggcctgatggtgctgagttgccattcaccattgacaagtaaagatct

SEQ ID NO: 25 - DNA, 2306 bp, Artificial Sequence
  Description of Artificial Sequence Synthetic polynucleotide
  GAG-GFP
gtcgacatgggtgctagagcttctgttttgtctggtggtgaattggatagatgggaaaagatcagattaagaccaggtggtaagaagaagtacaagttgaaacatatcgtttgggcctccagagaattggaaagatttgctgttaatccaggcttgttggaaacttctgaaggttgcagacaaatcttgggtcaattgcaaccatctttacaaaccggttctgaagaactgagatccttgtataatactgttgctaccttgtactgcgtccatcaaagaattgaaatcaaggataccaaagaggccttggacaagattgaagaagaacagaacaagtccaagaagaaggctcaacaagctgctgctgatactggtcattctaatcaagtttctcagaactacccaatcgtccaaaacattcaaggtcaaatggttcatcaagccatttctccaagaactttgaatgcatgggtcaaagttgtagaagaaaaggctttttcacctgaggttatcccaatgttttctgctttatctgaaggtgctactccacaagatttgaacactatgttgaacactgttggtggtcatcaagctgctatgcaaatgttgaaagaaaccattaacgaagaggctgctgaatgggatagagttcatccagttcatgctggtccaattgcacctggtcaaatgagagaacctagaggttctgatattgctggtactacttctaccttgcaagaacaaattggttggatgactcataacccaccaattccagttggtgaaatctacaagagatggatcattttgggcttgaacaagatcgtcagaatgtactctccaacctccattttggatattagacagggtccaaaagaaccattcagagattacgttgacagattctacaagaccttgagagctgaacaagcttctcaagaggttaagaattggatgaccgaaaccttgttggttcaaaatgctaatccagattgcaagaccatcttgaaagctttaggtccaggtgctacattggaagaaatgatgactgcttgtcaaggtgtaggtggtccaggtcataaggctagagttttggctgaagctatgtctcaagttactaatccagccaccattatgatccaaaagggcaactttagaaaccagagaaagaccgttaagtgcttcaactgtggtaaagaaggtcatattgctaagaactgtagagccccaagaaaaaaaggttgttggaagtgcggaaaagagggtcatcaaatgaaggattgcactgaaagacaagctaacttcttgggtaaaatttggccatctcataagggtagaccaggtaactttttacaatctagaccagaacctactgctccaccagaagaatcttttagatttggtgaagaaaccaccactccatctcaaaagcaagaacctatcgacaaagagttgtatccattggcttcattgagatctttgttcggttctgatccatcatctcaaggtggttctacatcttctgaattctgctctagaagatatagaggtcctggtattcatagaccagttgctactatggtttcaaagggtgaagaattattcactggtgttgttccaatcttggttgaattggacggtgatgttaatggtcacaagttttctgtttctggtgaaggtgagggtgatgctacttatggtaaattgactttgaagttcatctgcaccacaggtaaattgccagttccatggccaactttggttactactttgacttatggtgtccagtgcttttctagatacccagatcatatgaagcaacacgacttttttaagtccgctatgccagaaggttacgttcaagaaagaacgattttcttcaaggacgacggtaactacaaaactagagctgaagttaagttcgaaggtgataccttggttaacaggattgaattgaagggcatcgatttcaaagaggatggtaacatattgggccataagttggagtacaactacaactctcataacgtttacatcatggccgacaagcaaaagaacggtattaaggttaacttcaagatcaggcacaacatcgaagatggttctgttcaattggctgatcactatcaacaaaacaccccaattggtgatggtccagttttgttgccagataaccattacttgtctactcaatccgctttgtctaaggacccaaacgaaaaaagggatcacatggttttgttggaattcgttactgctgctggtattaccttaggtatggatgagttgtacaagtaagcggccgc

(19) United States
(12) Patent Application Publication
(10) Pub. No.: US 2022/0145309 A1
(43) Pub. Date: May 12, 2022

(54) Yeast-Based Oral Vaccination

(71) Applicant: ESPEROVAX INC., Plymouth, MI (US)

(72) Inventors: David James O'Hagan, Whitmore Lake, MI (US); Savita Sunil Nikam, Canton, MI (US); Roger S. Newton, Maple City, MI (US)

(21) Appl. No.: 17/612,179

(22) PCT Filed: May 21, 2020
(86) PCT No.: PCT/US2020/034094

§ 371 Date: Nov. 17, 2021

Related U.S. Application Data
(60) Provisional application No. 62/850,681, filed on May 21, 2019.
(60) Provisional application No. 62/991,536, filed on Mar. 18, 2020.

(51) Int. Cl. C12N 15/81; A61K 39/145; A61K 39/21

(52) U.S. Cl. CPC C12N 15/81; A61K 2039/523; A61K 39/21; A61K 39/145

(57) ABSTRACT

Various recombinant yeast suitable for use in oral vaccination, vaccine compositions, food compositions, methods of vaccinating an animal, and related methods, kits, and nucleic acid molecules are described.

### CROSS-REFERENCES TO RELATED APPLICATIONS

This applications claims the benefit of priority to U.S. Provisional Appl. No. 62/850,681, filed May 21, 2019, and U.S. Provisional Appl. No. 62/991,536, filed Mar. 18, 2020, the contents of which are hereby incorporated in the entirety and for all purposes.

### FIELD

The disclosure relates to the field of vaccination. More particularly, the disclosure relates to the field of yeast-based oral vaccination. Specific examples relate to recombinant yeast cells, vaccine compositions, food compositions, methods of producing a vaccine, and methods of vaccinating an animal. The disclosure also relates to various other methods, kits, and nucleic acid molecules.

### BACKGROUND

Vaccination is an inexpensive and effective method for reducing and preventing human disease. Unfortunately, though, global vaccination rates still leave about 15% of the world's population susceptible to preventable disease. Many conventional vaccines rely on injections administered by healthcare professionals, which inherently limits the number of patients that can be treated. Furthermore, injection-based vaccines often have significant storage and handling requirements, which can inhibit the scaling of production, delivery, and administration efforts.

Yeast based vaccine platforms have been described for high-volume vaccine production and/or use as an oral vaccination. See, U.S. Pat. No. 10,117,915. However, to-date, such platforms do not efficiently release complex immunogens such as virus like particles or enveloped virus like particles.

A need exists, therefore, for improved vaccines, vaccine components, vaccine compositions, and related methods, kits, and other vaccine-related technology.

### SUMMARY

The present inventors have unexpectedly discovered that regulated permeabilization of the cell wall in recombinant yeast cell that expresses an immunogen, e.g., a virus like particle or enveloped virus like particle, can significantly improve immunogen release by the recombinant yeast. Without wishing to be bound by theory, the present inventors hypothesize that, by inducing regulated permeabilization using one or more methods described herein, the amount of immunogen (e.g., in the form of a VLP comprising the immunogen and/or containing a packaged nucleic acid sequence encoding the immunogen) released can be improved as compared to a recombinant yeast cell that is not permeablized; the degree of recombinant yeast cell viability can be maintained at a higher level as compared to previously described permeabilization methods; the efficacy of the resulting vaccine composition can be increased; and/or immunogen release can be more selective as compared to previously described methods. One or more of these improvements, in turn, can significantly improve the amount or purity of immunogen recovered in an in vitro immunogen production method; the amount of immunogen provided to antigen presenting cells by the yeast; and/or the amount of immunogen released by a recombinant yeast cell in the gastrointestinal tract of a subject having been administered the recombinant yeast cell.

Regulated permeabilization can be induced by inducing expression of a cell wall permabilizing agent, such as a cell wall degrading enzyme (e.g., mannase, glucanase, chitinase, or combination thereof), inducing expression of an inhibitor of cell wall biosynthesis, or by reducing or eliminating expression of a component of the cell wall biosynthesis pathway in a regulated manner. Without wishing to be bound by theory, the present inventors further hypothesize that regulated induction of a cell wall degrading enzyme can, in addition to the unexpected benefits described above, unexpectedly increase the inherent adjuvant activity provided by yeast cell wall components in the vaccine composition by increasing the shedding, and/or presentation, of cell wall degradation products, such as yeast glycoproteins, beta glucan, or mannan.

In a preferred embodiment, the antigen presenting cells to which immunogen is delivered are in vivo, such as in the gastrointestinal tract of a subject that has been, e.g., orally, administered the recombinant yeast. In some cases, the yeast are administered orally as an oral vaccine. Various exemplary recombinant yeast cells suitable for use in oral vaccination are described herein.

In one aspect the recombinant yeast cell suitable for use in oral vaccination is derived from a wild-type yeast cell. Generally, the recombinant yeast cell is a host cell that comprises at least one heterologous nucleic acid sequence. For example, the heterologous element can comprise a heterologous promoter. The heterologous promoter can be a promoter from a cell of a different species or strain as compared to the host cell. In some embodiments, the heterologous promoter is a copy of an endogenous promoter that is operably linked to a second nucleic acid sequence, wherein the endogenous promoter and second nucleic acid sequence are not operably linked in a wild-type yeast cell.

In some embodiments, the heterologous promoter is an endogenous promoter that is present in a different location in a genome or intracellular location as compared to the wild-type location of the endogenous promoter. For example, the heterologous promoter can be in a different chromosomal location as compared to the chromosomal location of the promoter in a wild-type yeast cell. As another, example, the heterologous promoter can be present on a plasmid or episomal fragment, wherein the promoter is located in a host cell chromosome in a wild-type yeast cell. In some embodiments, the heterologous promoter can be a promoter that is naturally found in a different organelle as compared to the intracellular location of the heterologous promoter in the recombinant yeast cell. For example, a heterologous promoter can be a promoter from an endogenous yeast cell mitochondrial genome and operably linked to a second nucleic acid sequence in a nucleus of the recombinant yeast cell.

In one embodiment the recombinant yeast cell comprise a first nucleic acid sequence encoding a regulated promoter, a second nucleic acid sequence encoding an immunogen, and a third nucleic acid sequence encoding a cell wall degrading enzyme. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and/or third nucleic acid sequence is under control of the regulated promoter. In some embodiments, expression of the second and third nucleic acid sequences is under common genetic control of the regulated promoter.

In some embodiments, the immunogen is or comprises a component of a virus like particle (VLP), such as a capsid protein, or a functional fragment thereof. In some embodiments, the immunogen is or comprises a fusion protein comprising a first portion and a second portion, wherein the first portion comprises a capsid protein or functional fragment thereof and the second portion comprises an antigen. In some embodiments, the immunogen is or comprises a component of an enveloped VLP (eVLP), such as a matrix protein, or a functional fragment thereof. In some embodiments, the immunogen is or comprises a fusion protein comprising a first portion and a second portion, wherein the first portion comprises a matrix protein or functional fragment thereof and the second portion comprises an antigen. In some embodiments, the VLP comprises a fusion protein comprising a first portion and a second portion, wherein the first portion comprises a VLP forming unit (e.g., HIV-GAG or a capsid protein or a functional fragment thereof) and the second portion comprises an immunogen or a reporter polypeptide. In some cases, the reporter polypeptide is an enzyme. In some cases, the reporter polypeptide is a fluorescent protein. In embodiments, containing a reporter protein, such VLPs can be useful for tracking administration of VLPs to a subject and/or uptake of VLPs by cells of a subject.

An exemplary GAG-GFP fusion is set forth in SEQ ID NO. 14. In some cases, the GAG-GFP fusion comprises at least 25, 50, 100, 125, or 150 contiguous amino acids of, or all of, SEQ ID NO. 14. In some cases, the GAG-GFP fusion protein is at least 80%, 85%, 90%, 95%, or 99% identical to at least 25, 50, 100, 125, or 150 contiguous amino acids of, or all of, SEQ ID NO. 14. In some cases, the GAG-GFP fusion protein comprises no more than 1, 2, 4, or 5, single amino acid insertions, substitutions, and/or deletions of SEQ ID NO. 14. In some cases, the GAG-GFP fusion protein comprises no more than 1, 2, 4, or 5, single amino acid insertions, substitutions, and/or deletions of a contiguous amino acid region of SEQ ID NO. 14 of at least 25, 50, 100, 125, or 150 amino acids in length.

In some embodiments, the cell-wall degrading enzyme is a glucanase, such as a β-glucanase.

In one embodiment the recombinant yeast cell suitable for use in oral vaccination is derived from a wild-type yeast cell and comprises a first nucleic acid sequence encoding a regulated promoter, a second nucleic acid sequence encoding one or more proteins from an influenza virus (or coronavirus); and a third nucleic acid sequence encoding a cell wall degrading enzyme. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and/or third nucleic acid sequence is under control of the regulated promoter. In some embodiments, expression of the second and third nucleic acid sequences is under common genetic control of the regulated promoter. In some embodiments, the cell-wall degrading enzyme is a glucanase, such as a β-glucanaseo ra β-1,3-glucanase.

In some embodiments, the cell wall degrading enzyme is a glucanase, such as a β-glucanase.

In some cases, the proteins from an influenza virus are selected from the group consisting of M1 matrix protein (e.g., human flu M1 matrix protein) or a functional fragment thereof, hemagluttinin or an immunogenic fragment thereof, and neuraminidase or an immunogenic fragment thereof. In some cases, the second nucleic acid encodes at least two proteins from an influenza virus selected from the group consisting of M1 matrix protein (e.g., human flu M1 matrix protein) or a functional fragment thereof, hemagluttinin or an immunogenic fragment thereof, and neuraminidase or an immunogenic fragment thereof. In some cases, the second nucleic acid encodes human flu M1 matrix protein, hemagglutinin, and neuraminidase.

In some cases, the proteins from a coronavirus are selected from the group consisting of a coronavirus spike protein (e.g., COVID-19 spike protein), or an immunogenic or functional fragment thereof, and a coronavirus M1 matrix protein (e.g., COVID-19 M1 matrix protein) or an immunogenic or functional fragment thereof. In some cases, the second nucleic acid encodes at least two proteins from a coronavirus selected from the group consisting of M1 matrix protein or an immunogenic or functional fragment thereof, and coronavirus spike protein, or an immunogenic or functional fragment thereof.

A suitable COVID-19 spike protein can be, but is not limited to, an immunogenic fragment comprising the sequence set forth in SEQ ID NO. 22. In some cases, the COVID-19 spike protein comprises at least 25, 50, 100, 125, or 150 contiguous amino acids of, or all of, SEQ ID NO. 22. In some cases, the COVID-19 spike protein comprises a protein that is at least 80%, 85%, 90%, 95%, or 99% identical to at least 25, 50, 100, 125, or 150 contiguous amino acids of, or all of, SEQ ID NO. 22. In some cases, the COVID-19 spike protein comprises a protein comprising no more than 1, 2, 4, or 5, single amino acid insertions, substitutions, and/or deletions of SEQ ID NO. 22. In some cases, the COVID-19 spike protein comprises no more than 1, 2, 4, or 5, single amino acid insertions, substitutions, and/or deletions of a contiguous amino acid region of SEQ ID NO. 22 of at least 25, 50, 100, 125, or 150 amino acids in length.

The recombinant yeast cell can be a recombinant Saccharomyces cerevisiae cell.

In an exemplary embodiment the recombinant yeast cell suitable for use in oral vaccination is derived from a wild-type Saccharomyces cerevisiae yeast cell and comprises a first nucleic acid sequence encoding the Tet-off regulated promoter, a second nucleic acid sequence encoding one or more immunogens selected from the group consisting of Human Flu M1 matrix, hemagglutinin, and neuraminidase protein from an influenza virus; and a third nucleic acid sequence encoding a cell wall degrading enzyme. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and/or third nucleic acid sequence is under control of the regulated promoter. In some embodiments, expression of the second and third nucleic acid sequences is under common genetic control of the regulated promoter.

In an exemplary embodiment the recombinant yeast cell suitable for use in oral vaccination is derived from a wild-type Saccharomyces cerevisiae yeast cell and comprises a first nucleic acid sequence encoding the Tet-off regulated promoter, a second nucleic acid sequence encoding one or more coronavirus immunogens described herein; and a third nucleic acid sequence encoding a cell wall degrading enzyme. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and/or third nucleic acid sequence is under control of the regulated promoter. In some embodiments, expression of the second and third nucleic acid sequences is under common genetic control of the regulated promoter.

In another embodiment, the recombinant yeast cell suitable for use in oral vaccination is derived from a wild-type yeast cell and comprises a first nucleic acid sequence encoding a regulated promoter, a second nucleic acid sequence encoding an immunogen, and a third nucleic acid sequence encoding a cell wall inhibiting toxin. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and third nucleic acid sequences is under common genetic control of the regulated promoter. In some embodiments, expression of the second and/or third nucleic acid sequence is under control of the regulated promoter.

In one aspect, the present invention provides a plurality of any one of the foregoing recombinant yeast cells, or any one of the recombinant yeast cells described herein, or a combination thereof. The plurality can range from at least 1×10⁶ to about 1×10¹⁵ cells, or from at least 1×10⁷ to about 1×10¹⁴ cells, or from at least 1×10⁸ to about 1×10¹³ cells.

The plurality of recombinant yeast cells can be in a culture medium comprising a density of from about 1×10⁵ cells/mL to about 2×10⁹ cells/mL, preferably from about 1×10⁸ cells/mL to about 2×10⁹ cells/mL. The plurality of recombinant yeast cells can be in a concentrated liquid comprising a density of from about 1×10⁹ cells/mL to about 1×10¹⁰ cells/mL. For example, the liquid can be a concentrated culture medium or the plurality of recombinant yeast cells can be concentrated by separating the cells from a culture medium and resuspending the cells in a buffer. The plurality of cells can be a freeze dried or spray-dried composition. In some cases, the freeze dried composition comprises from at least 1×10⁶ cells/g to 1×10⁹ cells/g. In some cases, the spray-dried composition comprises from at least 1×10⁶ cells/g to 1×10⁹ cells/g.

Various exemplary vaccine compositions are also described herein.

In one embodiment the vaccine composition comprises an ingestible vessel defining a cavity, such as a capsule, and a recombinant yeast cell disposed in the cavity. The recombinant yeast cell disposed in the cavity can, e.g., be any one of the foregoing recombinant yeast cells, or any one of the recombinant yeast cells described herein, or a combination thereof, e.g., in a liquid, concentrated liquid, or a solid (e.g., freeze dried or spray dried) preparation. In some embodiments, the ingestible vessel comprises from at least 1×10⁶ recombinant yeast cells to about 1×10¹² recombinant yeast cells. In some embodiments, the recombinant yeast cell is derived from a wild-type yeast cell and comprises a first nucleic acid sequence encoding a regulated promoter, a second nucleic acid sequence encoding an immunogen, and a third nucleic acid sequence encoding a cell wall degrading enzyme. In some embodiments, expression of the second and third nucleic acid sequences can be under common genetic control of the regulated promoter. In some embodiments, expression of the second and/or third nucleic acid sequence is under control of the regulated promoter.

In another embodiment the vaccine composition comprises an ingestible vessel defining a cavity and a recombinant yeast cell disposed in the cavity. The recombinant yeast cell is derived from a wild-type yeast cell and comprises a first nucleic acid sequence encoding a regulated promoter, a second nucleic acid sequence encoding an immunogen, and a third nucleic acid sequence encoding a cell wall inhibiting toxin. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and third nucleic acid sequences can be under common genetic control of the regulated promoter. In some embodiments, expression of the second and/or third nucleic acid sequence is under control of the regulated promoter.

Another example vaccine composition comprises an ingestible capsule defining a cavity and a freeze-dried or spray-dried recombinant yeast cell derived from a wild-type yeast cell disposed in the cavity. The recombinant yeast cell can comprise a first nucleic acid sequence encoding a regulated promoter, a second nucleic acid sequence encoding a VLP immunogen, and a third nucleic acid sequence encoding a cell wall degrading enzyme. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and third nucleic acid sequences can be under common genetic control of the regulated promoter. In some embodiments, expression of the second and/or third nucleic acid sequence is under control of the regulated promoter.

Various food compositions are also described herein

In one embodiment the food composition comprises at least one foodstuff and at least one vaccine composition comprising an ingestible vessel defining a cavity and a recombinant yeast cell described herein or composition comprising a plurality of recombinant yeast cells described herein disposed in the cavity. In another embodiment the food composition comprises at least one foodstuff; and a plurality of vaccine compositions, each of which comprises a polymeric shell defining a cavity and a plurality of recombinant yeast cells described herein disposed in the cavity. In another embodiment the food composition comprises at least one foodstuff and a vaccine composition comprising a plurality of recombinant yeast cells described herein disposed in a cavity defined by a polymeric shell. In another example, a food composition comprises a matrix comprising at least one foodstuff and a vaccine composition comprising a plurality of recombinant yeast cells described herein. In some cases, the vaccine composition is admixed with the foodstuff matrix.

Another example vaccine composition comprises a plurality of recombinant yeast cells as described herein spray-dried in combination with alginate or chitosan, or a combination thereof and one or more excipients. Suitable excipients include, but are not limited to MgCl₂, CaCl₂, and combinations thereof. See, Szekalska et al., Materials (Basel). 2018, Sep. 11 (9):1522; and U.S. Pat. No. 9,700,519. The recombinant yeast cell can comprise a first nucleic acid sequence encoding a regulated promoter, a second nucleic acid sequence encoding a VLP immunogen, and a third nucleic acid sequence encoding a cell wall degrading enzyme or cell wall inhibitor toxin. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and third nucleic acid sequences can be under common genetic control of the regulated promoter. In some embodiments, expression of the second and/or third nucleic acid sequence is under control of the regulated promoter.

Various exemplary methods of producing a vaccine are also described.

In one embodiment the method of producing a vaccine comprises creating a recombinant yeast cell described herein by introducing into a wild-type yeast cell a first nucleic acid sequence encoding a regulated promoter, a second nucleic acid sequence encoding an immunogen, and a third nucleic acid sequence encoding a cell wall degrading enzyme or cell wall inhibitor and disposing the recombinant yeast cell in a cavity defined by an ingestible vessel. In some embodiments, the method comprises or further comprises culturing the recombinant yeast cell to produce a plurality of recombinant yeast cells. In some embodiments, the method further comprises harvesting at least a portion of the plurality of recombinant yeast cells and disposing the harvested recombinant yeast cells in a cavity defined by an ingestible vessel.

In another embodiment the method of producing a vaccine comprises creating a recombinant yeast cell described herein by introducing into a wild-type yeast cell a first nucleic acid sequence encoding a positive repressible promoter that is repressed in the presence of a repressor, a second nucleic acid sequence encoding an immunogen, and a third nucleic acid sequence encoding a cell wall degrading enzyme. In some embodiments, the method comprises or further comprises growing a plurality of recombinant yeast cells comprising the positive repressible promoter that is repressed in the presence of the repressor in a culture comprising the repressor. In some embodiments, the method comprises or further comprises disposing the plurality of recombinant yeast cells in a cavity defined by an ingestible vessel or spray drying the plurality of recombinant yeast cells with a polymeric medium such as alginate or chitosan.

In some embodiments, the method further comprises removing a sufficient amount of the repressor to promote the production of a protein encoded by the second and/or third nucleic acid sequence. The repressor can be removed by replacing, diluting, or removing, a culture media comprising the repressor. The repressor can be removed by diluting, or allowing a dilution of, the repressor, e.g., in a digestive tract of a subject.

In another embodiment the method of producing a vaccine comprises creating a recombinant yeast cell by introducing into a wild-type yeast cell a first nucleic acid sequence encoding a positive repressible promoter that is repressed in the presence of a repressor, a second nucleic acid sequence encoding an immunogen, and a third nucleic acid sequence encoding a cell wall degrading enzyme. In some embodiments, the method comprises or further comprises growing a plurality of the recombinant yeast cells comprising the positive repressible promoter in a culture comprising the repressor. In some embodiments, the method comprises or further comprises freeze-drying or spray-drying the plurality of recombinant yeast cells; and disposing the plurality of recombinant yeast cells in a cavity defined by an ingestible vessel. At least one, or each of the first nucleic acid sequence, second nucleic acid sequence, and third nucleic acid sequences comprising a nucleic acid sequence that does not occur naturally in the wild-type yeast cell. Expression of the second and third nucleic acid sequences can be under common genetic control of the regulated promoter. In some embodiments, expression of the second and/or third nucleic acid sequence is under control of the regulated promoter.

In some embodiments, the method further comprises removing a sufficient amount of the repressor to promote the production of a protein encoded by the second and/or third nucleic acid sequence. The repressor can be removed and then the plurality of recombinant yeast cells can be incubated for a pre-defined period of time such that the positive repressible promoter is activated and expression of the second and/or third nucleic acid sequences occurs. The promoter activation can be achieved prior to freeze drying. Additionally or alternatively, promoter activation can be performed after or by freeze drying and/or reconstitution of the plurality of freeze-dried recombinant yeast cells. Additionally or alternatively, promoter activation can be performed after or by administering the plurality of, e.g., freeze-dried and optionally reconstituted, recombinant yeast cells.

Various exemplary methods of vaccinating an animal are also described herein.

In one embodiment the method of vaccinating an animal comprises orally delivering to said animal a vaccine composition according to an embodiment. In another embodiment the method of vaccinating an animal comprises orally delivering to said animal a food composition according to an embodiment. In some embodiments the animal is a human, and the method comprises instructing said human to orally ingest a vaccine composition according to an embodiment.

In another embodiment the method comprises instructing said human to orally ingest a food composition according to an embodiment.

Various methods of supplying a vaccine are also described herein.

In one embodiment the method of supplying a vaccine comprises producing a plurality vaccine compositions, each vaccine composition of the plurality of vaccine compositions comprising a vaccine composition according to an embodiment; and delivering the plurality of vaccine compositions to an individual designated for delivering individual vaccine compositions of the plurality of vaccine compositions to individual animals of said plurality of animals for the purpose of vaccinating individual animals of said plurality of animals.

In another embodiment the method of supplying a vaccine comprises producing a plurality food compositions, each food composition of the plurality of food compositions comprising a food composition according to an embodiment; and delivering the plurality of food compositions to an individual designated for delivering individual food compositions of the plurality of food compositions to individual animals of said plurality of animals for the purpose of vaccinating individual animals of said plurality of animals.

Various exemplary kits are also described herein.

In one embodiment the kit comprises a packaging substrate, a vaccine composition according to an embodiment, and instructions for using the vaccine composition.

In another embodiment the kit comprises a packaging substrate, a food composition according to an embodiment, and instructions for using the food composition.

Various exemplary isolated nucleic acid molecules are also provided herein.

In one embodiment the isolated nucleic acid molecule comprises SEQ ID NO. 1, which encodes for an exemplary secreted beta-glucanase cell wall degrading enzyme useful in the methods, compositions, and kits of the present invention. In another embodiment the isolated nucleic acid molecule comprises SEQ ID NO. 2, which encodes for an exemplary secreted H1N1 influenza A hemaglutinin useful in the methods, compositions, and kits of the present invention. In another embodiment, the isolated nucleic acid molecule comprises SEQ ID NO. 3, which encodes for an exemplary H1N1 influenza A matrix protein 1. In another embodiment the isolated nucleic acid molecule comprises SEQ ID NO. 4, which encodes for an exemplary secreted chitinase cell wall degrading enzyme useful in the methods, compositions, and kits of the present invention.

In another embodiment, the isolated nucleic acid molecule encodes for SEQ ID NO. 15 or a functional or immunogenic fragment thereof. In some cases, the isolated nucleic acid molecule encodes for at least 25, 50, 100, 125, or 150 contiguous amino acids of, or all of, SEQ ID NO. 15. In some cases, the isolated nucleic acid molecule encodes for a protein that is at least 80%, 85%, 90%, 95%, or 99% identical to at least 25, 50, 100, 125, or 150 contiguous amino acids of, or all of, SEQ ID NO. 15. In some cases, the isolated nucleic acid encodes for a protein that comprises no more than 1, 2, 4, or 5, single amino acid insertions, substitutions, and/or deletions of SEQ ID NO. 15. In some cases, the isolated nucleic acid molecule encodes a protein comprising no more than 1, 2, 4, or 5, single amino acid insertions, substitutions, and/or deletions of a contiguous amino acid region of SEQ ID NO. 15 of at least 25, 50, 100, 125, or 150 amino acids in length.

In another embodiment, the isolated nucleic acid molecule encodes for SEQ ID NO. 16 or a functional or immunogenic fragment thereof. In some cases, the isolated nucleic acid molecule encodes for at least 25, 50, 100, 125, or 150 contiguous amino acids of, or all of, SEQ ID NO. 16. In some cases, the isolated nucleic acid molecule encodes for a protein that is at least 80%, 85%, 90%, 95%, or 99% identical to at least 25, 50, 100, 125, or 150 contiguous amino acids of, or all of, SEQ ID NO. 16. In some cases, the isolated nucleic acid encodes for a protein that comprises no more than 1, 2, 4, or 5, single amino acid insertions, substitutions, and/or deletions of SEQ ID NO. 16. In some cases, the isolated nucleic acid molecule encodes a protein comprising no more than 1, 2, 4, or 5, single amino acid insertions, substitutions, and/or deletions of a contiguous amino acid region of SEQ ID NO. 16 of at least 25, 50, 100, 125, or 150 amino acids in length.

In another embodiment, the isolated nucleic acid molecule encodes for SEQ ID NO. 17 or a functional or immunogenic fragment thereof. In some cases, the isolated nucleic acid molecule encodes for at least 25, 50, 100, 125, or 150 contiguous amino acids of, or all of, SEQ ID NO. 17. In some cases, the isolated nucleic acid molecule encodes for a protein that is at least 80%, 85%, 90%, 95%, or 99% identical to at least 25, 50, 100, 125, or 150 contiguous amino acids of, or all of, SEQ ID NO. 17. In some cases, the isolated nucleic acid encodes for a protein that comprises no more than 1, 2, 4, or 5, single amino acid insertions, substitutions, and/or deletions of SEQ ID NO. 17. In some cases, the isolated nucleic acid encodes a protein comprising no more than 1, 2, 4, or 5, single amino acid insertions, substitutions, and/or deletions of a contiguous amino acid region of SEQ ID NO. 17 of at least 25, 50, 100, 125, or 150 amino acids in length.

In another embodiment the isolated nucleic acid molecule encodes a cell wall inhibiting toxin. In some cases, the cell wall inhibiting toxin comprises at least 25, 50, 100, 125, or 150 contiguous amino acids of, or all of, SEQ ID NO. 23. In some cases, the toxin comprises no more than 1, 2, 3, 4, or 5 single amino acid substitutions, deletions, and/or additions relative to the protein encoded by SEQ ID NO. 5 or the polypeptide sequence set forth in SEQ ID NO. 23. In some cases, the toxin is at least 80%, 85%, 90%, 95%, or at least 99%, identical to a secreted protein sequence encoded by SEQ ID NO. 5. In some cases, the β-1-3-glucanase is at least 80%, 85%, 90%, 95%, or at least 99% identical to at least 25, 50, 100, or 125, contiguous amino acids of, or all of, SEQ ID NO. 23. In some cases, the nucleic acid encodes the cell wall inhibiting toxin encoded by SEQ ID NO. 5. In some cases, the exemplary cell wall inhibiting toxin comprises SEQ ID NO. 23.

Additional understanding of the invention, including the example recombinant yeast cells suitable for use in oral vaccination, vaccine compositions, food compositions, methods of producing a vaccine, methods of vaccinating an animal, and related methods, kits, and nucleic acid molecules, can be obtained by reviewing the detailed description of selected examples, below, and the appended drawings.

### BRIEF DESCRIPTION OF THE DRAWINGS

FIG. 1 is a schematic representation of an example recombinant yeast cell.

FIG. 2 is a schematic representation of an example vaccine composition.

FIG. 3 is a schematic representation of an example food composition.

FIG. 4 is a schematic representation of an example method of producing a vaccine.

FIG. 5 is a schematic representation of another example method of producing a vaccine.

FIG. 6 is a schematic representation of an example method of vaccinating an animal.

FIG. 7 is a schematic representation of another example method of vaccinating an animal.

FIG. 8 is a schematic representation of another example method of vaccinating an animal.

FIG. 9 is a schematic representation of another example method of vaccinating an animal.

FIG. 10 is a schematic representation of an example method of supplying a vaccine.

FIG. 11 is a schematic representation of an example method of supplying a vaccine.

FIG. 12 is a schematic representation of an example kit.

FIG. 13 is a schematic representation of another example kit.

FIG. 14 illustrates a Western blot staining of human influenza H1, M1, and N1 proteins obtained from eVLPs harvested from yeast cell culture medium.

FIG. 15 illustrates a schematic of eVLPs that carry an mRNA payload for expression of a recombinant immunogen in target cells of an administered host that take up (e.g., phagocytose) the eVLPs.

FIG. 16 illustrates RT-PCR results of eVLPs using EGFP-specific primers demonstrating presence of packaged EGFP mRNA in the eVLPs.

FIG. 17 illustrates a fluorescence microscope image of recombinant yeast cells secreting eVLPs carrying an EGFP mRNA payload.

FIG. 18 illustrates dendritic cells (left) expressing mRNA encoded EGFP from phagocytosed eVLPs (right).

FIG. 19 illustrates transmission electron micrographs of eVLPs obtained from culture media.

FIG. 20 illustrates a method of producing alginate-encapsulated yeast cell microspheres for oral administration.

FIG. 21 illustrates fluorescence microscope images of alginate-encapsulated yeast cell microspheres. Left: illustrates bulk material after microsphere encapsulation. Right: illustrates a single microsphere encapsulating a plurality of VLP producing recombinant yeast cells.

FIG. 22 illustrates results of oral administration of alginate-encapsulated yeast cell microspheres. Bars indicate from left to right: anti-GAG-GFP serum antibody levels in mice administered a high dose of oral alginate-encapsulated yeast cell microspheres; anti-influenza serum antibody levels in mice administered standard H1/N1 injectable vaccine; anti-GAG-GFP serum antibody levels in mice administered saline control; anti-GAG-GFP serum antibody levels in mice administered a medium (M) dose of oral alginate-encapsulated yeast cell microspheres; and anti-GAG-GFP serum antibody levels in mice administered a low (L) dose of oral alginate-encapsulated yeast cell microspheres.

FIG. 23 illustrates an exemplary construct encoding an HIV GAG-GFP fusion under the control of an ADH2 promoter and a beta-glucanase (Egress 1) under the control of an ADH2 promoter.

FIG. 24 illustrates an exemplary construct encoding an HIV GAG-MS2 fusion protein, a beta-glucanase, and a nucleic acid sequence encoding an mRNA that encodes EGFP and includes multiple MS2-protein binding sites.

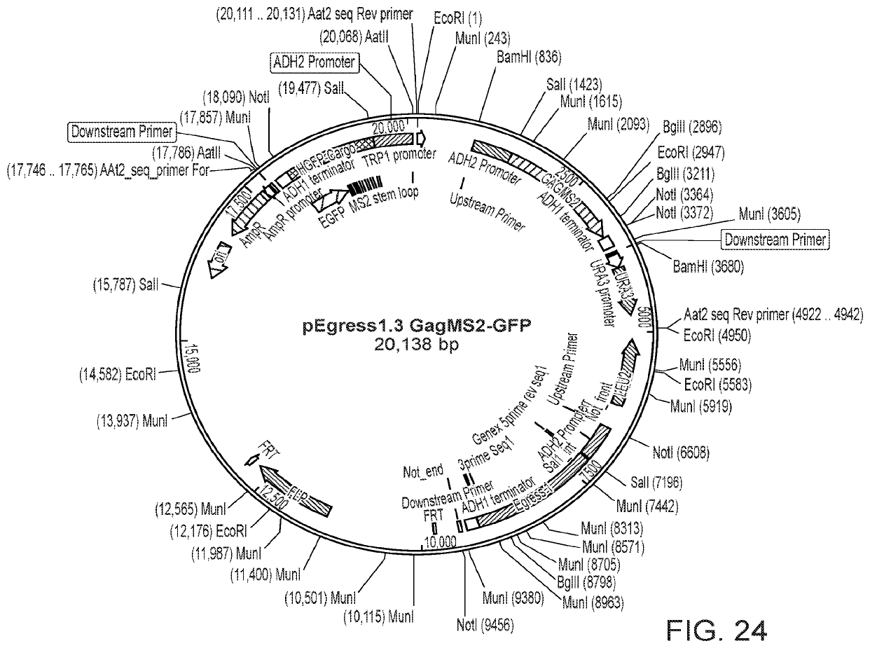

FIG. 25 illustrates an exemplary sequence of a nucleic acid sequence encoding an influenza neuraminidase protein.

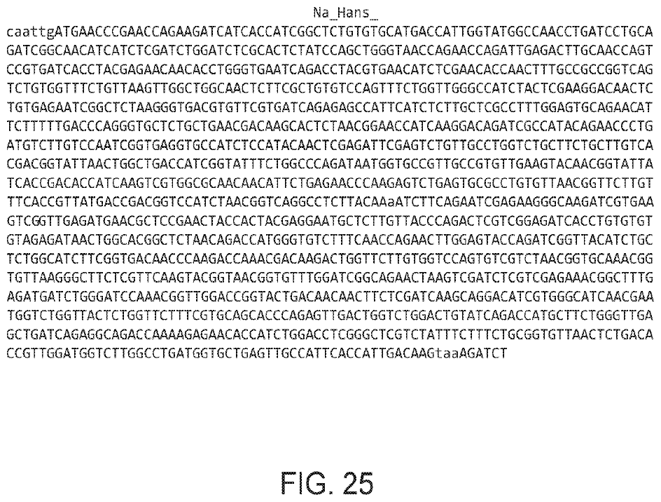

FIG. 26 illustrates an exemplary sequence of a nucleic acid sequence encoding an HIV GAG-GFP fusion protein.

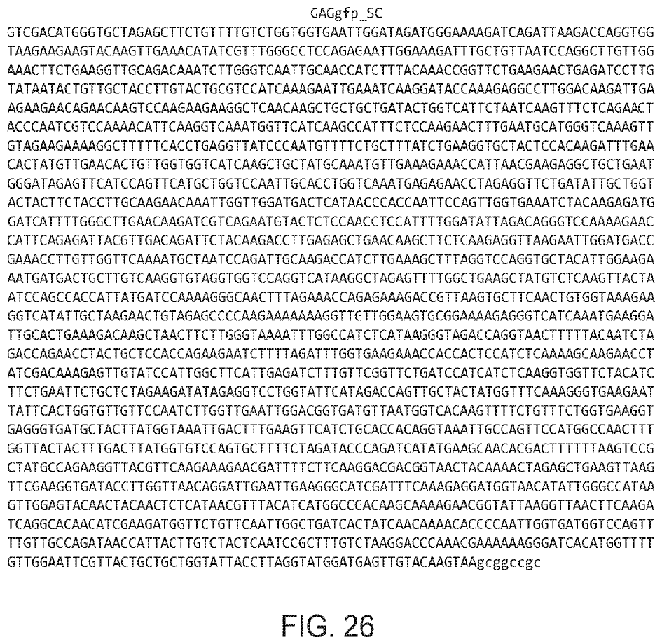

FIG. 27 illustrates a vector for expression of Ebola glycoprotein (GP) under the control of an ADH2 promoter in yeast. Ebola VP40 can also be expressed under the control of a different or a common promoter.

FIG. 28 illustrates results of expressing Ebola GP with yeast secretory signal and zymolase. Expression of GP in recombinant yeast cells was induced and samples were taken at the indicated time points.

FIG. 29 illustrates DAPI staining of yeast cells with and without glucanase treatment. DAPI staining indicates permeabilization of the yeast cell wall.

FIG. 30 illustrates Ebola VP40 expression in supernatant and yeast cell lysate after 6 or 24 hours induction of VP40 and glucanase by glucose starvation.

FIG. 31 illustrates VP40 expression results after induction of expression in yeast by glucose starvation. XXXXX represents glucanase.

FIG. 32 illustrates certain combinations of heterologous proteins for VLP production with influenza or Ebola antigens.

FIG. 33 illustrates an influenza VLP tricassette expression vector for yeast cell expression.

FIG. 34 illustrates an Ebola VLP tricassette expression vector for yeast cell expression.

FIG. 35 illustrates yeast intracellular expression levels of Ebola VLPs containing GP and VP40 under conditions in which a cell wall permeabilizing agent is co-expressed.

FIG. 36 illustrates yeast secreted (supernatant) expression levels of Ebola VLPs containing GP and VP40 under conditions in which a cell wall permeabilizing agent is co-expressed.

FIG. 37 illustrates yeast secreted (supernatant) expression levels of influenza VLPs containing M1 and H1 under conditions in which a cell wall permeabilizing agent is co-expressed.

FIG. 38 illustrates yeast intracellular expression levels of influenza VLPs containing GP and VP40 under conditions in which a cell wall permeabilizing agent is co-expressed.

FIG. 39 illustrates yeast secreted (supernatant) expression levels of influenza VLPs under conditions in which a cell wall permeabilizing agent is co-expressed.

FIG. 40 illustrates a schematic diagram of an expression cassette for incorporating a gene of interest, e.g., yGFP into a VLP by including an MS2 binding site array. RNA transcripts containing the gene of interest and MS2 binding site array can be packaged into VLPs that are at least partially formed from a fusion protein containing an MS2 binding protein sequence.

### DETAILED DESCRIPTION

The following detailed description and the appended drawings describe and illustrate various example recombinant yeast suitable for use in producing a vaccine immunogen. Such recombinant yeast cells can be used, e.g., for oral vaccination, for making vaccine compositions, in methods of vaccinating an animal, and in related methods, kits, and nucleic acid molecules. The description and drawings are provided to enable one skilled in the art to make and use one or more recombinant yeast suitable for use in oral vaccination, vaccine compositions, kits, and nucleic acid molecules and to perform the example methods. They are not intended to limit the scope of the claims in any manner.

As used herein, the term “animal” refers to a vertebrate. The term includes mammals, birds, fish, reptiles, and amphibians. As such, the term includes humans, domesticated pets, such as dogs and cats, feral cats, horses, cattle, and other vertebrate animals. The term also includes agriculturally important animals such as domesticated pigs, chickens, cows, sheep, goats, horses, donkeys, mules, ducks, geese and turkeys.

As used herein, the term “cavity” refers to an open space defined by an object. On its own, the term does not require any specific structure or physical properties and includes, for example, spaces with exposed openings and enclosed spaces.

As used herein, the term “common genetic control” a property of multiple nucleic acid sequences being regulated by the same promoter. The term includes nucleic acid arrangements in which the multiple nucleic acid sequences are positioned downstream of a single promoter that regulates the expression of both nucleic acid sequences. The term also includes nucleic acid arrangements in which one of the multiple nucleic acid sequences is positioned downstream of a first copy of the promoter and another of the multiple nucleic acid sequences is positioned downstream of a second copy of the promoter. It should be appreciated, that where multiple copies of a promoter are used in a scheme for expression of proteins under common genetic control, the copies need not be identical in sequence and minor variations in promoter sequence are tolerated so long as functional equivalency is maintained.

As used herein, the term “ingestible” refers to the ability of a referenced element to be ingested by an animal.

As used herein, the term “regulated promoter” refers to a region of DNA that initiates transcription of a particular gene under specific conditions. The term includes inducible promoters and repressible promoters. Examples of inducible promoters include both positive inducible promoters, i.e., inducible promoters that are activated in the presence of the inducer, such as by interaction between the inducer and an activator molecule to enable binding of the combined entity to the inducible promoter to effect transcription of downstream genes controlled by the inducible promoter, and negative inducible promoters, i.e., inducible promoters that are activated in the presence of the inducer, such as by interaction between the inducer and a repressor to block or disable binding of the repressor to the inducible promoter, thereby removing suppression of transcription of downstream genes controlled by the inducible promoter. Examples of repressible promoters include both positive repressible promoters, i.e., promoters that are repressed in the presence of the repressor, such as by interaction between the repressor and an activator molecule to block or disable binding of the activator molecule to the repressible promoter, thereby removing activation of transcription of downstream genes controlled by the repressible promoter, and negative repressible promoters, i.e., promoters that are repressed in the presence of the repressor, such as by interaction between the repressor and a corepressor molecule to enable binding of the combined entity to the repressible promoter to effect transcription of downstream genes controlled by the repressible promoter. The term also includes promoters that can be regulated as both a positive inducible promoter and a negative inducible promoter, and promoters that respond to environmental queues, such as the presence or absence of light, the absence of a particular molecule, and any other promoter that can be specifically regulated by providing or removing a particular molecule or environmental queue.

As used herein, the term “vessel” refers to a structure capable of partially or completely containing a substance, such as one or more recombinant yeast cells. On its own, the term does not require any specific structure or physical properties and includes, for example, open structures, closed structures, single component structures, multi-component structures, rigid structures, and flexible structures.

As used herein, the term “virus like particle” or “VLP” refers to a non-infectious nanostructures composed of viral structural proteins and lacking viral nucleic acid. A virus like particle morphologically resembles a virus, hut, without more, lacks the ability to infect a host cell. VLPs are typically comprised of at least one structural component, such as a capsid or matrix protein that forms a particle shell.

As used herein, the term “enveloped virus like particle” or “eVLP” refers to a virus like particle that includes host cell-derived membranes, typically a lipid-based membrane obtained during the budding process as the virus emerges from the host cell. eVLPs typically comprise at least one matrix protein. In some cases, an eVLP may comprise 2 or 3, or more, different matrix proteins. In some cases, one or more of the matrix proteins are engineered to display an antigenic peptide sequence on the outer surface of the protein shell of the eVLP. For example, antigenic peptide sequence can be inserted into one or more matrix protein loop sequences such that the antigenic peptide sequence exposed on the outer protein surface of an assembled eVLP. In some cases, the eVLP is engineered to include one or more immunogenic peptides on the surface of the eVLP. In some cases, the eVLP component(s) are expressed in a host cell that further expresses one or more antigen (e.g., glycopeptides) that can embed in the lipid bilayer envelope of the eVLP.

As used herein, the term “non-enveloped virus like particle” refers to a VLP that does not include a host-cell derived membrane. The acronym neVLP refers to the term “non-enveloped virus like particle.” neVLPs can comprise at least one capsid protein. In some cases, an neVLP may comprise 2 or 3, or more, different capsid proteins. In some cases, one or more of the capsid proteins are engineered to display an antigenic peptide sequence on the outer surface of the neVLP. For example, antigenic peptide sequence can be inserted into one or more capsid protein loop sequences such that the antigenic peptide sequence is exposed on the outer protein surface of an assembled neVLP.

VLPs, including neVLPs and/or eVLPs can be engineered to include a nucleic acid binding peptide, which in turn can bind a specific nucleic acid binding site sequence. As described further below, one exemplary nucleic acid binding peptide is found in the MS2 coat protein, which binds an, e.g., 19-nucleotide, ribosomal binding site of the MS2 replicase mRNA, which folds into a hairpin loop structure. Typically one or more nucleic acid binding sites are included as a repeated array of nucleic acid binding sites to increase the amount of cognate protein localized to the nucleic acid. In some cases, the repeated sequence can compromise genetic stability of the recombinant coding sequence. In one embodiment, the nucleic acid binding sites in the repeated array are synonymous binding sites that are different in sequence and yet retain the cognate protein binding function. Such arrays of synonymous nucleic acid binding sites are described in, e.g., Wu et al., Genes Dev. 2015 Apr. 15 (29(8); 876-886.

Such VLPs engineered to include a nucleic acid binding peptide can be used to deliver a nucleic acid encoding an antigen to an antigen presenting cell to increase a vaccine response by expressing the antigen in the antigen presenting cell. In one embodiment, the VLP comprises at least one capsid or matrix protein fused to the nucleic acid binding element. In an exemplary embodiment, the VLP comprises an HIV-Gag-MS2 fusion, such as the GAG-MS2 fusion set forth in SEQ ID NO. 13. In some cases, the GAG-MS2 fusion protein comprises at least 25, 50, 100, 125, or 150 contiguous amino acids of, or of, SEQ ID NO. 13. In some cases, the GAG-MS2 fusion protein is at least 80%, 85%, 90%, 95%, or 99% identical to at least 25, 50, 100, 125, or 150 contiguous amino acids of, or of, SEQ ID NO. 13. In some cases, the GAG-MS2 fusion protein comprises no more than 1, 2, 4, or 5, single amino acid insertions, substitutions, and/or deletions of SEQ ID NO. 13. In some cases, the GAG-MS2 fusion protein comprises no more than 1, 2, 4, or 5, single amino acid insertions, substitutions, and/or deletions of a contiguous amino acid region of SEQ ID NO. 13 of at least 25, 50, 100, 125, or 150 amino acids in length.

Alternative fusions for binding a nucleic acid include, but are not limited to a fusion of influenza or coronavirus matrix protein M1 or M2 to a nucleic acid binding peptide, a fusion of a coronavirus spike protein to a nucleic acid binding peptide, and a fusion of HBV nucleocapsid protein to a nucleic acid binding peptide.

Additionally, or alternatively, such VLPs can be used to deliver a nucleic acid encoding a reporter to increase a reporter signal by expressing the reporter in a cell that takes up the VLP.

Alternative nucleic acid binding peptides and corresponding nucleic acid binding site sequences, include but are not limited to those described in U.S. 2017/0233762, the contents of which are herein incorporated by reference in the entirety for all purposes including but not limited to RNA ligand sequences and RNA binding peptide sequences and their use. A skilled person will appreciate that multiple RNA binding peptide sequences (e.g., in a VLP fusion protein) and their ligands (e.g., in the target nucleic acid to be packaged) can be incorporated to package multiple copies of the same nucleic acid or to package multiple different nucleic acids.

Where polypeptide sequences are disclosed herein, e.g., by sequence listing, it is understood that such polypeptides can include an N-terminal secretion signal suitable to support secretion of a mature-form (e.g., wherein the signal sequence is cleaved) polypeptide from a host organism such as a yeast cell. Where a signal peptide is already present in the disclosed sequence, a skilled person will appreciate that such a sequence also discloses the mature form of the polypeptide after cleavage of the signal peptide. Moreover, a skilled person will appreciate that a signal sequence can be replaced with a signal sequence optimized for a host organism described herein.

Described herein are methods and compositions that provide improved recombinant immunogen release by regulated permeabilization of a recombinant yeast cell that produces said recombinant immunogen. As described herein in various embodiments, this improved immunogen release can be provided by regulated induction of expression of a cell wall degrading enzyme, regulated repression of expression of a component of a cell wall biosynthesis pathway, or regulated induction of expression of an inhibitor of cell wall biosynthesis.

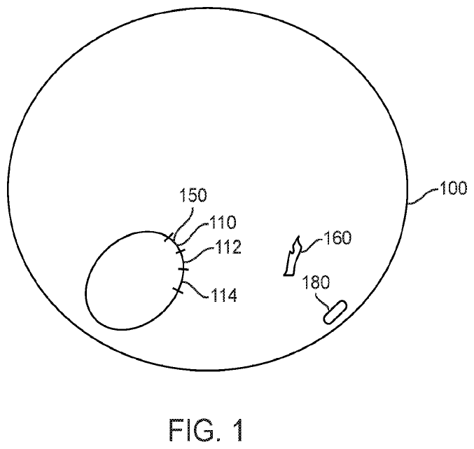

FIG. 1 is a schematic representation of an example recombinant yeast cell 100. The recombinant yeast cell 100 comprises a first nucleic acid sequence 110 encoding a regulated promoter 150; a second nucleic acid sequence 112 encoding an immunogen 160, preferably a VLP immunogen; and a third nucleic acid sequence 114 encoding a cell wall degrading enzyme 180. In some embodiments, at least one, or each, of the first nucleic acid sequence 110, second nucleic acid sequence 112, and third nucleic acid sequence 114 comprises a nucleic acid sequence that does not occur naturally in the wild-type yeast cell and that has been artificially introduced into the wild-type yeast cell to produce recombinant yeast cell 100. In some cases, expression of the second nucleic acid sequence 112 and/or the third nucleic acid sequence 114 are under common genetic control of the regulated promoter 150. Accordingly, the recombinant yeast cell 100 has been genetically modified to include at least one immunogen gene 112, at least one cell wall-degrading enzyme gene 114, and at least one regulated promoter 110. The first, second, and/or third nucleic acid sequences can be present on one or more plasmids. In some cases, at least one, or all of the first, second, and third nucleic acid sequences are inserted into the genome of the yeast cell at the same, or at a different, locus. In a preferred embodiment, the immunogen is, or is a component of, a VLP, such as an eVLP.

The recombinant yeast cell 100 can be produced from any suitable wild-type yeast cell and a skilled artisan will be able to select a wild-type yeast cell for producing a recombinant yeast cell according to a particular embodiment based on various considerations, including the nature of the cell wall, immunogen, and cell wall degrading enzyme to be used in the particular embodiment, the availability of the wild-type yeast cell, the relative ease with which the wild-type yeast cell can be transformed with a vector or vectors comprising the first, second, and third nucleic acid sequences, the relative ease with which the wild-type yeast cell can be grown in production level quantities, and the length of time over which the wild type yeast cell remains stable after being freeze-dried or processed using other techniques to achieve suspension of growth and other activities. Examples of suitable wild type yeast cells include Saccharomyces cerevisiae (S. cerevisiae, also known as “baker's yeast”), Pichia pastoris, and Hansenula polymorpha.

The inventors have determined that S. cerevisiae is useful as a wild-type yeast cell in production of a recombinant yeast cell according to embodiments of the invention at least because of its ready availability, well-characterized transformation effectiveness, and well-characterized handling techniques. The inventors have identified S. cerevisiae strain Sc1602 MAT alpha, ura3-, leu-, pep4-, och1- as a useful wild-type yeast cell in production of a recombinant yeast cell according to embodiments of the invention.

The first nucleic acid sequence 110 encodes a regulated promoter 150. The regulated promoter 150 can comprise any suitable regulated promoter and a skilled artisan will be able to select a regulated promoter for a recombinant yeast cell according to a particular embodiment based on various considerations, including the nature of the wild-type yeast cell used in the production of the recombinant yeast cell, any desired type of control over the production of the immunogen and/or cell wall degrading enzyme, and any equipment and/or supplies needed to control expression of the VLP immunogen and cell wall degrading enzyme using a particular inducible promoter. Examples of suitable regulated promoters include inducible promoters, including positive inducible promoters, negative inducible promoters, and inducible promoters that can be regulated as both a positive inducible promoter and a negative inducible promoter, and repressible promoters, including positive repressible promoters, negative repressible promoters, and repressible promoters that can be regulated as both a positive repressible promoter and a negative repressible promoter. Examples of suitable regulated promoters include the Gall inducible promoter, which activates transcription of genes controlled by the promoter in the presence of galactose, and the ADH2 promoter, which activates transcription in the absence of glucose. Other examples of regulated promoters considered suitable include, but are not limited to, PTet, pTP1, pTEF1, pPYK1, pADH1, FMD1, pHXT7, pGAL1, pGAL7, pGAL10, pPHO5, pCUP1, and pDAN1.

The inventors have determined that the Tet-off regulated promoter, a positive repressible promoter, is particularly advantageous for inclusion as the regulated promoter in recombinant yeast cells according to the invention. In the Tet-oft system, transcription of genes controlled by the regulated promoter is turned off when tetracycline or one of its derivatives is present. The inventors consider the inclusion of this regulated promoter particularly advantageous at least because of the production methods it enables. For example, as described in detail below, inclusion of this regulated promoter in a recombinant yeast cell enables a method in which a culture of recombinant yeast cells is grown in a laboratory environment in the presence of tetracycline or a tetracycline derivative. During this stage of the method, the genes controlled by the Tet-off system in the recombinant yeast cells in the culture, such as the nucleic acid sequence that encodes an immunogen and/or the nucleic acid sequence that induces cell wall permeabilization, such as a cell wall degrading enzyme, are not transcribed. The tetracycline or tetracycline derivative can be removed at a later time. For example, a sufficient amount of repressor can be removed for a predefined period of time after a culture of the cells has achieved a sufficient density or growth phase in the culture, thereby activating transcription of the nucleic acid sequence that encodes the immunogen and/or the nucleic acid sequence that encodes a cell wall degrading enzyme for the length of the predefined period of time. This enables production of a desired amount of immunogen and/or cell wall degrading enzyme prior to harvesting the recombinant yeast cells in the culture. In turn, this ensures that, when the recombinant yeast cells are ingested by a patient to be vaccinated, such as during an oral vaccination in which the patient ingests freeze-dried recombinant yeast, an amount of immunogen and/or cell wall degrading enzyme are available immediately, which can positively impact the efficacy of the vaccine.

As another example, a positive repressible promoter can be used to regulate cell wall permeability by regulated repression of a cell wall biosynthesis pathway. For example, a recombinant yeast cell can be engineered to include a positive repressible promoter operably linked to a component of a cell wall biosynthesis pathway and to express an immunogen, e.g., in a regulated fashion. The recombinant yeast cell can be cultured under conditions to permit cell wall biosynthesis and then subsequently cell wall biosynthesis can be repressed by removal of the repressor. In some embodiments, the regulated repression of a cell wall biosynthesis pathway is provided by promoter replacement or insertion of a positive repressible promoter operably linked to an endogenous component of a cell wall biosynthesis pathway. Alternatively, an endogenous cell wall biosynthesis pathway component can be knocked out and an alternate, e.g., copy, introduced into the recombinant yeast cell that is operably linked to a positive repressible promoter.

As described herein, in some embodiments, the immunogen and cell-wall permeabilizing agent (e.g., cell wall degrading enzyme, cell wall biosynthesis toxin, etc.) are under the common genetic control of a regulatable promoter. Alternatively, in some embodiments, the immunogen and cell-wall permeabilizing agent are differentially regulated. In some embodiments, the regulated promoter is operably linked to the nucleic acid sequence encoding the cell wall permeabilizing agent. In some embodiments, a different, e.g., regulated, promoter is operably linked to the nucleic acid sequence encoding the immunogen or a component thereof.

In some cases, the promoter operably linked to the cell-wall permeabilizing agent is selected to induce or de-repress expression of the cell wall permeabilizing agent after the recombinant yeast have been cultured to a sufficient density (e.g., 1×10⁸ cells-mL, OD₆₀₀≥10, or OD₆₀₀≥20) or growth phase (e.g., log phase, mid-log phase, or late-log phase growth). In some cases, the promoter operably linked to the cell-wall permeabilizing agent is selected to induce or de-repress expression of the cell wall permeabilizing agent after the recombinant yeast have been harvested or after the recombinant yeast have been administered to a subject.

In some cases, the promoter operably linked to the immunogen or component thereof is selected to induce or de-repress expression of the immunogen prior to administration of the recombinant yeast to a subject. For example, immunogen production can be de-repressed or induced during culture of the recombinant yeast cells. In some methods of the present invention, immunogen expression is induced or de-repressed and then expression of the permeabilizing agent is induced or de-repressed. In some cases, the yield of expressed immunogen can be enhanced by inducing expression of cell wall permeabilizing agent after induction of immunogen expression. In other cases, e.g., where inefficient release of immunogen overwhelms the secretory capacity of the host cell, it may be preferable to induce expression of the cell wall permeabilizing agent prior to, or at the same time, as inducing the expression of the immunogen. As described herein, one exemplary method for simultaneous induction of both immunogen and cell wall permeabilization agent is to operably link the nucleic acid sequences encoding both the immunogen and the permeabilization agent to a regulatable common genetic control element.

In some embodiments, the nucleic acid sequence encoding the cell-wall permeabilizing agent is under control of a regulated promoter and the nucleic acid sequence encoding the immunogen is constitutively expressed.

In some cases, methods for producing recombinant VLPs in a permeabilized yeast further include inhibiting cell replication during the induction phase. In some embodiments, the inhibition of replication can improve VLP production by reducing the metabolic burden of replication. Cell replication can be inherently inhibited by inhibiting cell wall production (e.g., using a Killer Toxin) inhibiting cell wall maintenance (e.g., using a cell wall degrading enzyme), or inhibiting genome replication, inducing expression of a checkpoint activator, such as TEL1 or Mps1.

In some cases, genome replication is inhibited by inhibiting expression or activity of endogenous DNA polymerase, in some cases, DNA polymerase is inhibited by removing all or part of the genomic region encoding the endogenous yeast DNA polymerase. In some cases, methods of producing VLPs described herein include inducing expression of a recombinant recombinase, such as CRE recombinase, and thereby inducing recombination at one or more, preferably two loxP sites in the genome at the genomic region encoding the endogenous DNA polymerase. Typically, the loxP sites are positioned to flank an essential region of the endogenous DNA polymerase. In some embodiments, the CRE recombinase is under the genetic control of a regulatable promoter that is common to a nucleic acid sequence encoding a cell-wall permeabilizing agent and/or a nucleic acid sequence encoding an immunogen or a component thereof (e.g., an influenza hemagglutinin or neuraminidase, or a coronaviral spike protein), and/or a nucleic acid sequence encoding a VLP forming protein sequence, such as a GAG protein (e.g., HIV Gag), a matrix protein (e.g., influenza M), a nucleocapsid protein (e.g., coronavirus N or influenza NP), an envelope protein (e.g., coronavirus E), or a combination thereof. Thus, in some embodiments, yeast host cells described herein contain one or more recombination sites, such as loxP sites at or flanking a DNA polymerase encoding genomic region, and a nucleic acid encoding a heterologous recombinase, such as a. CRE recombinase.

A recombination based approach for inhibiting cell replication can be particularly advantageous in forming a vaccine that suitable for administration to a mammalian subject, wherein the vaccine contains, or is likely to contain, at least a portion of whole yeast cells because such cells will not replicate. For example, in some embodiments, VLPs described herein are induced with simultaneous or sequential recombination to inhibit replication, e.g., with simultaneous or sequential permeabilization, cell culture supernatant containing VLPs are collected and used to form a vaccine. In some cases, cell culture supernatant used to form a vaccine further contains a yeast, cell component, which can, e.g., provide an adjuvant, effect. Additionally, or alternatively, in some embodiments described herein, the VLPs are from cell culture supernatant and non-replicable yeast cells are also harvested and admixed with formulation agents to produce the vaccine.

The second nucleic acid sequence 112 encodes an immunogen 160, preferably a VLP immunogen. The immunogen can comprise, e.g., any suitable VLP immunogen and a skilled artisan will be able to select a VLP immunogen for a recombinant yeast cell according to a particular embodiment based on various considerations, including the identity and antigens of any specific disease-causing agent for which the recombinant yeast cell is being produced for inclusion in a vaccine, such as the various vaccine compositions described herein, the overall size of the VLP immunogen, and other considerations. Also, while the illustrated embodiment shows a single nucleic acid sequence 112 encoding a single immunogen, it is noted that multiple nucleic acid sequences that each encode an immunogen, or a component thereof, can be included in a recombinant yeast cell according to an embodiment. Examples of suitable numbers of nucleic acid sequences that each encode an immunogen include, but are not limited to, one, at least one, more than one, two, a plurality, three, four, five, six, seven, eight, nine, ten, and more than ten. Also, the second nucleic acid sequence 112 can encode a naked VLP immunogen or an enveloped VLP immunogen.

Examples of suitable VLP immunogens include, but are not limited to, one or more immunogens from Cholera, Dengue, Diphtheria, Hepatitis A, Hepatitis B, Hepatitis E, emophilus influenzae type b (Hib), Human papillomavirus (HPV), Influenza, Japanese encephalitis, Malaria, Measles, Meningococcal meningitis, Mumps, Pertussis, Pneumococcal disease, Poliomyelitis, Rabies, Rotavirus, Rubella, Tetanus, Tick-borne encephalitis, Tuberculosis, Typhoid, Varicella, Yellow Fever, Campylobacter jejuni, Chagas Disease, Chikungunya, Dengue, terotoxigenic Escherichia coli, Enterovirus 71 (EV71), Group B Streptococcus (GBS), Herpes Simplex Virus, HIV-1, Human Hookworm Disease, Leishmaniasis Disease, Malaria, Nipah Virus, ntyphoidal Salmonella Disease, Norovirus, Paratyphoid fever, spiratory Syncytial Virus (RSV), Schistosomiasis Disease, Shigella, Staphylococcus aureus, Streptococcus pneumoniae, Streptococcus pyrogenes, Tuberculosis, and Universal Influenza Vaccine.

Inclusion of nucleic acid sequences that encode the eVLP immunogens Human Flu M1 matrix, hemagglutinin, and neuraminidase proteins from an influenza virus is considered advantageous for a recombinant yeast cell according to the invention that is intended for use in an oral vaccine against the influenza virus.

The third nucleic acid sequence 114 can encode a cell wall permeabilizing agent such as a cell wall degrading enzyme 180. The cell wall degrading enzyme can comprise any suitable cell wall degrading enzyme and a skilled artisan will be able to select a cell wall degrading enzyme for a recombinant yeast cell according to a particular embodiment based on various considerations, including the nature and size of the immunogen encoded by the second nucleic acid sequence 112, the number of different nucleic acids that encode immunogens included in the recombinant yeast cell, the nature of the cell wall of the recombinant yeast cell, and other considerations. Examples of suitable cell wall degrading enzymes include, but are not limited to, a glucanase enzyme such as a β-1,3-glucanase, a mannanase enzyme, a chitinase, and other enzymes capable of degrading a yeast cell wall.

In some cases, the β-1,3-glucanase comprises at least 25, 50, 100, 125, or 150 contiguous amino acids of, or all of, SEQ ID NO. 10. In some cases, the β-1,3-glucanase comprises no more than 1, 2, 3, 4, or 5 single amino acid substitutions, deletions, and/or additions relative to the protein encoded by SEQ ID NO. 1 or the polypeptide sequence set forth in SEQ ID NO. 10. In some cases, the β-1-3-glucanase is at least 80%, 85%, 90%, 95%, or at least 99%, identical to a secreted protein sequence encoded by SEQ ID NO. 1. In some cases, the β-1-3-glucanase is at least 80%, 85%, 90%, 95%, or at least 99% identical to at least 25, 50, 100, 125, or 150 contiguous amino acids of, or all of, SEQ ID NO. 10. In some cases, the glucanase comprises no more than 1, 2, 4, or 5, single amino acid insertions, substitutions, and/or deletions of a contiguous amino acid region of SEQ ID NO. 10 of at least 25, 50, 100, 125, or 150 amino acids in length.

In some cases, the mannanase comprises at least 25, 50, 100, 125, or 150 contiguous amino acids of, or all of, SEQ ID NO. 11. In some cases, the mannanase comprises no more than 1, 2, 3, 4, or 5 single amino acid substitutions, deletions, and/or additions relative to the polypeptide sequence set forth in SEQ ID NO. 11. In some cases, the mannanase is at least 80%, 85%, 90%, 95%, or at least 99%, identical to a secreted protein sequence encoded by SEQ ID NO. 11. In some cases, the β-1-3-glucanase is at least 80%, 85%, 90%, 95%, or at least 99% identical to at least 25, 50, 100, 125, or 150 contiguous amino acids of, or all of, SEQ ID NO. 11. In some cases, the mannanase comprises no more than 1, 2, 4, or 5, single amino acid insertions, substitutions, and/or deletions of a contiguous amino acid region of SEQ ID NO. 11 of at least 25, 50, 100, 125, or 150 amino acids in length.

In some cases, the chitinase comprises at least 25, 50, 100, 125, or 150 contiguous amino acids of, or all of, SEQ ID NO. 12. In some cases, the chitinase comprises no more than 1, 2, 3, 4, or 5 single amino acid substitutions, deletions, and/or additions relative to the protein encoded by SEQ ID NO. 4 or the polypeptide sequence set forth in SEQ ID NO. 12. In some cases, the chitinase is at least 80%, 85%, 90%, 95%, or at least 99%, identical to a secreted protein sequence encoded by SEQ ID NO. 4. In some cases, the chitinase is at least 80%, 85%, 90%, 95%, or at least 99% identical to at least 25, 50, 100, 125, or 150 contiguous amino acids of, or all of, SEQ ID NO. 12. In some cases, the chitinase comprises no more than 1, 2, 4, or 5, single amino acid insertions, substitutions, and/or deletions of a contiguous amino acid region of SEQ ID NO. 12 of at least 25, 50, 100, 125, or 150 amino acids in length.

In some embodiments, a primary component of the recombinant yeast cell wall is β-1,3-glucans, and the cell wall degrading enzyme is or comprises a β-1,3-glucanase. In some embodiments, a primary component of the recombinant yeast cell wall is mannan, and the cell wall degrading enzyme is or comprises a mannanase. In some embodiments, a primary component of the recombinant yeast cell wall is chitin, and the cell wall degrading enzyme is or comprises a chitinase. In some embodiments, expression of a combination of one or two, or more, glucanase cell wall degrading enzymes, such as a β-1-3-glucanase and a β-1-6-glucanase, is induced to permeabilize the cell wall in a regulated manner. In some embodiments, expression of a combination of one or two, or more, chitinase cell wall degrading enzymes is induced to permeabilize the cell wall in a regulated manner. In some embodiments, expression of a combination of one or two, or more, mannanase cell wall degrading enzymes is induced to permeabilize the cell wall in a regulated manner. In some embodiments, expression of a combination of two or more of, or each of, a glucanase, a chitinase, and mannase are induced to permeabilize the cell wall in a regulated manner.

In some embodiments, the cell wall permeabilizing agent is a cell wall degrading enzyme from a yeast that is a natural predator of the host cell. For example, certain spore-forming ascomycetous yeasts of the genera Pichia and Williopsis express cell wall degrading enzymes that exhibit high glucosidic activity against intact S. cerevisiae cell walls. Accordingly, in certain embodiments, the cell wall permeabilizing agent can be a Pichia or Williopsis cell wall degrading enzyme. As another example, certain bacteria such as Arthrobacter or Cellulosimicrobium cellulans express cell wall degrading enzymes that exhibit high glucosidic activity against intact S. cerevisiae cell walls. Accordingly, in some embodiments, the cell wall permeabilizing agent is a cell wall degrading enzyme from Arthrobacter or Cellulosimicrobium cellulans.

Inclusion of a nucleic acid sequence that encodes β-glucanase, such as β-1,3-glucanase, is considered particularly advantageous in recombinant yeast cells that include influenza-based VLP immunogens, such as the eVLP immunogens Human Flu M1 matrix, hemagglutinin, and neuraminidase proteins from an influenza virus as described above, at least because this particular cell wall degrading enzyme is expected to effectively degrade the yeast cell wall to a sufficient degree to allow the assembled eVLP to escape from the recombinant yeast cell.

In addition to, or as an alternative to inclusion of a nucleic acid sequence that encodes a cell wall degrading enzyme, a nucleic acid that encodes a cell wall inhibiting toxin can be included. In these embodiments, the encoded toxin inhibits or prevents formation of cell wall in newly formed recombinant yeast cells. As a result, newly-formed recombinant yeast cells lack a cell wall completely or have only a partially formed cell wall. In either scenario, the immunogen(s) included in the recombinant yeast cell are able to leave the newly-formed recombinant yeast cell without the aid of a cell wall degrading enzyme.

If included in a recombinant yeast cell according to an embodiment, a nucleic acid sequence encoding any suitable cell wall inhibiting toxin can be included. Examples of suitable cell wall inhibiting toxins include, but are not limited to, Williopsis mrakii killer toxin.

The first nucleic acid sequence 110, accordingly, can comprise any nucleic acid sequence that encodes the regulated promoter selected for the recombinant yeast cell according to a particular embodiment. Similarly, the second nucleic acid sequence 112 can comprise any nucleic acid sequence that encodes the immunogen, or immunogens, selected for the recombinant yeast cell according to a particular embodiment. Lastly, the third nucleic acid sequence 114 can comprise any nucleic acid sequence that encodes a cell wall permeabilizing agent, such as a cell wall degrading enzyme, selected for the recombinant yeast cell according to a particular embodiment.

Expression of the second nucleic acid sequence 112 and the third nucleic acid sequence 114 can be under common genetic control of the regulated promoter 150. In some embodiments, a genetic construct is made that includes each of the second nucleic acid sequence 112 and the third nucleic acid sequence positioned downstream from the first nucleic acid sequence. In other embodiments, the second nucleic acid sequence 112 is positioned downstream of, and under the genetic control of, a first copy of the first nucleic acid sequence 110 and the third nucleic acid sequence 114 is positioned downstream of, and under the genetic control of, a second copy of the first nucleic acid sequence 110. In the latter embodiments, the first copy of the first nucleic acid sequence 110 and the second nucleic acid sequence 112 can be positioned on the same or a different nucleic acid molecule (e.g., vector, plasmid, or chromosome) as the second copy of the first nucleic acid sequence 110 and the third nucleic acid sequence 114. For example, to produce a recombinant yeast cell according to one of these embodiments, a wild type yeast cell can be transformed with two different genetic vectors—a first genetic vector that encodes the first copy of the first nucleic acid sequence 110 and the second nucleic acid sequence 112, and a second genetic vector that encodes the second copy of the first nucleic acid sequence 110 and the third nucleic acid sequence 114.

In certain embodiments, the second nucleic acid sequence 112 encodes a fusion protein comprising a viral structural element and a nucleic acid binding protein. In these embodiments, the recombinant yeast cell 100 includes a fourth nucleic acid sequence 116 that encodes a binding target for the nucleic acid binding protein and an antigen or immunogen of interest. Similar to the first 110, second 112, and third 114 nucleic acid sequences, the fourth nucleic acid sequence 116 in these embodiments can comprise a nucleic acid sequence that does not occur naturally in the wild-type yeast cell and that has been artificially introduced into the wild-type yeast cell to produce recombinant yeast cell 100.

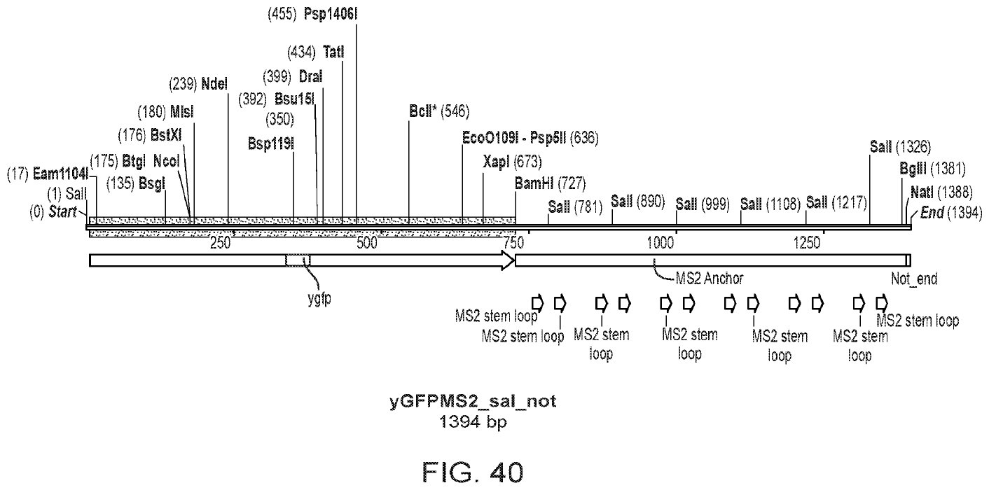

In one particular example in accordance with these embodiments, the second nucleic acid sequence encodes a Gag-MS2 fusion protein. The Gag portion of the fusion protein is an HIV gag protein that assembles to form viral particles while the MS2 portion of the fusion protein is an MS2 bacteriophage coat protein that naturally interacts with well-defined non-translated stem loop structures in RNA. An example of a suitable nucleic acid sequence for the second nucleic acid sequence in these embodiments comprises SEQ ID NO. 6. An example precursor to a suitable nucleic acid sequence for the fourth nucleic acid sequence in these embodiments comprises SEQ ID NO. 7, which is schematically illustrated in the last figure presented in FIG. 40. SEQ ID NO. 7 encodes the MS2 anchor, which includes a series of stem loop structures that MS2 protein can bind, and ygfp, which includes a series of well-characterized restriction enzyme sites that can be used for insertion of one or more selected antigens and/or immunogens. Accordingly, the sequence encoding the yGFP reporter can be substituted for any one of the antigens and/or immunogens described herein, including but not limited to a protein encoding a viral immunogen such as an influenza hemagluttinin and/or neuraminidase, or a coronaviral spike protein.

In some cases, the MS2 binding sequence can be a part of a repeated array of MS2 sequences. In some cases, the repeated MS2 sequences can compromise genetic stability of the recombinant coding sequence. In one embodiment, the nucleic acid binding sites in the repeated array are synonymous binding sites that are different in sequence and yet retain the cognate protein binding function. Such arrays of synonymous nucleic acid binding sites are described in, e.g., Wu et al., Genes Dev. 2015 Apr. 15 (29(8); 876-886. In some embodiments the MS2 sequence comprises a following hairpin loop forming sequence of SEQ ID NO. 8 (NRNDSASSANCASSSNNYN), wherein S represents C or G; D represents A, G, or U; R represents A or G; and Y represents C or U. In some embodiments, the nucleic acid binding sites are in a repeated array comprising from 8 to 48 iterations of an MS2 sequence, such as an MS2 sequence of SEQ ID NO. 8. In some embodiments, the repeated array comprises from 8 to 24, preferably 24 iterations of an MS2 sequence, such as an MS sequence of SEQ ID NO. 8. In some cases, the repeated array of nucleic acid binding sites is encoded by SEQ ID NO. 9.

These embodiments are considered particularly advantageous at least because the (e.g., Gag)-MS2 fusion protein works to bind and package the RNA corresponding to the antigen or immunogen encoded by the fourth nucleic acid sequence. In use, a recombinant yeast cell according to one of these embodiments will release VLPs that include RNA that encodes the antigen and/or immunogen of interest. If included in a vaccine composition or a food composition according to an embodiment, for example, the recombinant yeast cell will release VLPs that are ingested by dendritic cells or other antigen-presenting cells within the animal ingesting the vaccine composition or food composition. These cells can then translate the RNA and present the antigen(s) and/or immunogen(s) in the normal functioning of the animal's immune system, which may trigger a desired immune response to the selected antigen(s) and/or immunogen(s).

It will be appreciated that the GAG protein encoding sequence can be substituted with a variety of VLP forming protein sequences, including but not limited to a sequence encoding an influenza matrix protein or a coronaviral capsid protein, and the like.

In these embodiments, the fourth nucleic acid sequence can be, but need not be, under common genetic control of the regulated promoter 150 along with the first 110, second 112, and/or third 114 nucleic acid sequences. Also in some of these embodiments, the fourth nucleic acid sequence 114 can encode suitable antigen(s) or immunogen(s) of interest. Suitable examples include one or more viral antigens and/or immunogens, one or more bacterial antigens and/or immunogens, and any other antigen or immunogen considered suitable for eliciting an immune response. Antigens derived from influenza virus, RSV, HIV, and other viruses are considered particularly suitable for inclusion in these embodiments.

In some embodiments, the second nucleic acid sequence 112 encodes one or more viral genes sufficient to form VLPs while lacking one or more viral genes necessary to produce viral progeny. In some of these embodiments, the recombinant yeast cell 100 includes a fourth nucleic acid sequence 116 that encodes an antigen or immunogen of interest. Similar to the first 110, second 112, and third 114 nucleic acid sequences, the fourth nucleic acid sequence 116 in these embodiments can comprise a nucleic acid sequence that does not occur naturally in the wild-type yeast cell and that has been artificially introduced into the wild-type yeast cell to produce recombinant yeast cell 100.

VLPs, including neVLPs and/or eVLPs can be engineered to include an amplifiable replicon, or constructs encoding such a VLP. As used herein, an “amplifiable replicon” comprises minimal nucleic acid sequence(s) capable of supporting self-replication in a host cell. For example, a VLP can package an RNA nucleic acid that encodes an RNA-dependent RNA polymerase (RdRp) capable of replicating the packaged RNA nucleic acid or portion thereof at least 1, preferably at least 2 times. In some cases, the packaged nucleic acid includes a 5′ and/or a 3′ untranslated region (UTR), preferably the packaged nucleic acid includes a 5′ and a 3′ UTR. Typically, the amplifiable replicon contains a gene of interest, such as nucleic acid sequence encoding an immunogen.

In some embodiments, such amplifiable replicons can be constructed from portions of a parainfluenza virus (PIV) genome, such as a PIV type 5 (e.g., PIV5) genome. In some embodiments, the amplifiable replicon is a nucleic acid comprising, all, a functional portion of, or at least a portion of, a parainfluenza virus (e.g., PIV5) NP, V/P, and L gene, and optionally a gene of interest, such as nucleic acid sequence encoding an immunogen, an MS2 protein, an MS2 binding site, and/or a reporter. In some embodiments, the amplifiable replicon lacks one or more PIV genes (e.g., PIV5 genes) selected from the group consisting of M, F, SH and HN, or is incapable of expressing one or more of the PIV5 proteins selected from the group consisting of M, F, SH and HN. In some cases, the amplifiable replicon comprises PIV5 NP, V/P and L genes. In some cases, the amplifiable replicon comprises a gene of interest inserted between a PIV (e.g., PIV5) V/P and L gene.

Suitable PIV-based replicons include, but are not limited to, those replicons described in Wei et al., npj Vaccines 2, 32 (2017), preferably wherein said replicons include a nucleic acid encoding an immunogen (e.g., hemagluttinin, neuraminidase, or spike protein, or, reporter, and/or other gene of interest as described herein, an MS2 or other anchor sequence as described herein, an MS2 protein as described herein, or a VLP forming polypeptide as described herein (e.g., matrix, GAG, or capsid protein, or functional fragment thereof), between a 5′ and 3′ UTR. Such replicons can include, or be used in conjunction with other genetic elements, such as promoters or cis- or trans-acting helper polypeptides or genes, that are essential for supporting self-replication, as described in Wei et al., U.S. Pat. No. 9,034,343, and/or WO 2002/077211, or an orthologue thereof, such as an ortholog of an element or polypeptide of U.S. Pat. No. 9,034,343, that is disclosed in WO 2002/077211.

In an exemplary embodiment a replicon is generated by replacing PIV5 fusion glycoprotein (e.g., SEQ ID NO. 18), M, SH, and/or HN with a gene of interest, optionally wherein the replicon further includes a selectable marker (e.g., a hygromycin resistance marker), preferably wherein the selectable marker is inserted between V/P and L.

In an exemplary embodiment, the PIV5 L gene encodes a protein comprising SEQ ID NO. 19. In some cases, the PIV5 L gene encodes a protein comprising at least 25, 50, 100, 125, or 150 contiguous amino acids of, or all of, SEQ ID NO. 19. In some cases, the PIV5 L gene encodes a protein that is at least 80%, 85%, 90%, 95%, or 99% identical to at least 25, 50, 100, 125, or 150 contiguous amino acids of, or all of, SEQ ID NO. 19. In some cases, the PIV5 L gene encodes a protein comprising no more than 1, 2, 4, or 5, single amino acid insertions, substitutions, and/or deletions of SEQ ID NO. 19. In some cases, the PIV5 L gene encodes a protein comprising no more than 1, 2, 4, or 5, single amino acid insertions, substitutions, and/or deletions of a contiguous amino acid region of SEQ ID NO. 19 of at least 25, 50, 100, 125, or 150 amino acids in length.

In an exemplary embodiment, the PIV5 NP gene encodes a protein comprising SEQ ID NO. 20. In some cases, the PIV5 NP gene encodes a protein comprising at least 25, 50, 100, 125, or 150 contiguous amino acids of, or all of, SEQ ID NO. 20. In some cases, the PIV5 NP gene encodes a protein that is at least 80%, 85%, 90%, 95%, or 99% identical to at least 25, 50, 100, 125, or 150 contiguous amino acids of, or all of, SEQ ID NO. 20. In some cases, the PIV5 NP gene encodes a protein comprising no more than 1, 2, 4, or 5, single amino acid insertions, substitutions, and/or deletions of SEQ ID NO. 20. In some cases, the PIV5 NP gene encodes a protein comprising no more than 1, 2, 4, or 5, single amino acid insertions, substitutions, and/or deletions of a contiguous amino acid region of SEQ ID NO. 20 of at least 25, 50, 100, 125, or 150 amino acids in length.

In an exemplary embodiment, the PIV5 V/P gene encodes a protein comprising SEQ ID NO. 21. In some cases, the PIV5 V/P gene encodes a protein comprising at least 25, 50, 100, 125, or 150 contiguous amino acids of, or all of, SEQ ID NO. 21. In some cases, the PIV5 V/P gene encodes a protein that is at least 80%, 85%, 90%, 95%, or 99% identical to at least 25, 50, 100, 125, or 150 contiguous amino acids of, or all of, SEQ ID NO. 21. In some cases, the PIV5 V/P gene encodes a protein comprising no more than 1, 2, 4, or 5, single amino acid insertions, substitutions, and/or deletions of SEQ ID NO. 21. In some cases, the PIV5 V/P gene encodes a protein comprising no more than 1, 2, 4, or 5, single amino acid insertions, substitutions, and/or deletions of a contiguous amino acid region of SEQ ID NO. 21 of at least 25, 50, 100, 125, or 150 amino acids in length.

In some cases, one or all of the M, F, SH and HN PIV (e.g., PIV5) genes are replaced with the gene of interest. In some cases, one or more of NP, or V/P, are replaced with an influenza or coronaviral matrix or capsid protein gene or functional and/or immunogenic fragment thereof. In some cases, the amplifiable replicon comprises a nucleic acid sequence encoding an immunogenic and/or functional fragment of an influenza or coronaviral matrix or capsid protein. In some embodiments, the amplifiable replicon comprise a PIV (e.g., PIV5) 5′ and/or 3′ UTR. In some embodiments, the gene of interest and the RdRp gene are between the 5′ and 3′ UTRs. In some embodiments, the RdRp gene and the gene of interest are encoded as a single polypeptide that includes a self-cleaving peptide sequence between RdRp protein and the protein encoded by the gene of interest. In some cases, the self-cleaving peptide is a 2A self-cleaving peptide, such as a T2A peptide of Thosea asigna virus. Additional embodiments of amplifiable PIV5 replicons are described in, e.g., WO 2016/176510.

In some embodiments, an amplifiable replicon comprises a functional fragment, or all, of an RdRp gene from Nodamura virus (NoV). See, e.g., Biddlecome et al., PLoS One. 2019; 14(6): e0215031. In some cases, the amplifiable replicon comprises a NoV RdRp gene or functional fragment thereof and a gene of interest between, 5′ and 3′ UTRs of NoV RNA1. In some embodiments, the RdRp gene and the gene of interest are encoded as a single polypeptide that includes a self-cleaving peptide sequence between RdRp protein and the protein encoded by the gene of interest. In some cases, the self-cleaving peptide is a 2A self-cleaving peptide, such as a T2A peptide of Thosea asigna virus.

In some embodiments, an amplifiable replicon comprises a gene of interest and functional fragment, or all, of an alphaviral replicase. Alphaviruses encode four nonstructural proteins (nsP1-4), initially produced as a polyprotein P1234. nsP4 is the core RNA-dependent RNA polymerase but all four nsPs, or at least functional fragments thereof, are required for RNA synthesis. In some embodiments, an amplifiable replicon comprising a functional fragment, or all, of an alphaviral replicase, further comprises an alphaviral 5′ cis-acting element and/or a 3′ UTR, preferably a 5′ cis-acting element and a 3′ UTR. Suitable alphavirus replicon embodiments include, but are not limited to, those described in U.S. 2006/0198854, by Pushko, preferably wherein said replicons include a nucleic acid encoding an immunogen (e.g., hemagluttinin, neuraminidase, or spike protein, or, reporter, and/or other gene of interest as described herein, an MS2 or other anchor sequence as described herein, an MS2 protein as described herein, or a VLP forming polypeptide as described herein (e.g., matrix, GAG, or capsid protein, or functional fragment thereof), between the 5′ cis-acting element (e.g., 5′ UTR) and 3′ end of the replicon (e.g., 3′ UTR). Such replicons can include, or be used in conjunction with other genetic elements, such as promoters or cis- or trans-acting helper polypeptides or genetic elements, that are essential for supporting self-replication, as described in U.S. 2006/0198854.

Amplifiable replicon embodiments described herein, including those comprising one or more PIV genes and/or one or more replicase or RdRp genes (e.g., PIV5 or NoV RdRp or alphaviral replicase) can be packaged into any one of the VLPs described herein. Similarly, amplifiable replicons can be produced and packaged in any one of the yeast host cell systems described herein and released as VLPs from a permeabilized yeast host cell.

In one particular example in accordance with these embodiments, the second nucleic acid sequence 112 comprises at least a portion of each of Parainfluenza 5 (PIV5) NP, V/P, and L genes. In this example, the second nucleic acid sequence 112 lacks one or more of the PIV5 genes selected from the group consisting of M, F, SH, and HN.

In these embodiments, the fourth nucleic acid sequence can be, but need not be, under common genetic control of the regulated promoter 150 along with the first 110, second 112, and third 114 nucleic acid sequences. Also in these embodiments, the fourth nucleic acid sequence 114 can encode suitable antigen(s) or immunogen(s) of interest. Suitable examples include one or more viral antigens and/or immunogens, one or more bacterial antigens and/or immunogens, and any other antigen or immunogen considered suitable for eliciting an immune response. Antigens derived from influenza virus, RSV, HIV, and other viruses are considered particularly suitable for inclusion in these embodiments.

Following transformation, a recombinant yeast cell according to an embodiment can be treated further using any desirable and/or suitable techniques, processes, and/or methods based on a desired outcome, characteristic, or property. For example, as described in detail below, the recombinant yeast cell can be used in vaccine compositions. For these embodiments, the inventors have determined that dehydrated recombinant yeast cells are particularly advantageous. Accordingly, recombinant yeast cells according to a particular embodiment can be processed using conventional methods for dehydrating yeast, such as freeze-drying. Freeze-drying the recombinant yeast cell is considered particularly advantageous as the resulting freeze-dried recombinant yeast cell has a desirable residual moisture level and long-term stability. Accordingly, in some embodiments, the recombinant yeast cell comprises a freeze-dried recombinant yeast cell. Furthermore, in some embodiments, the recombinant yeast cell is microencapsulated and baked in food or placed in liquid.

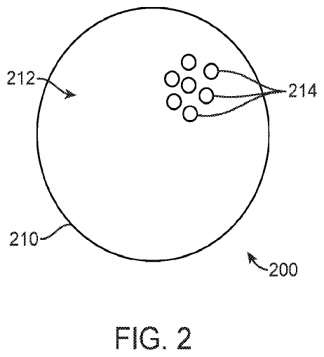

FIG. 2 is a schematic representation of an example vaccine composition 200. The vaccine composition 200 comprises an ingestible vessel 210 defining a cavity 212 and at least one recombinant yeast cell 214 according to an embodiment of the invention disposed in the cavity 212. For example, in an embodiment, the vaccine composition 200 includes at least one recombinant yeast cell that is genetically modified to produce an immunogen and a cell wall permeabilizing agent, e.g., under common genetic control of a regulated promoter. In some cases, the cell permeabilizing agent is a cell wall degrading enzyme. As another example, in an embodiment, the vaccine composition 200 includes at least one recombinant yeast cell that is genetically modified to produce an immunogen and a cell wall-inhibiting toxin, e.g., under common genetic control of a regulated promoter. Note that, in FIG. 2, none of the immunogen, the cell wall permeabilizing agent, regulated promoter, or the nucleic acids sequences that encode these elements are illustrated.

The ingestible vessel 210 can comprise any suitable ingestible vessel and a skilled artisan will be able to select an appropriate ingestible vessel for inclusion in a vaccine composition according to a particular embodiment based on various considerations, including the nature and quantity of the recombinant yeast cells included in the vaccine compositions, any storage and handling requirements, and other considerations. Examples of suitable ingestible vessels include, but are not limited to, capsules, acid-resistant capsules, and capsules defining pores.

The at least one recombinant yeast cell 214 can comprise any recombinant yeast cell according to an embodiment of the invention, including the example recombinant yeast cells described herein. Furthermore, the at least one recombinant yeast cell 214 can comprise any suitable number of recombinant yeast cells, and a skilled artisan will be able to select an appropriate number of recombinant yeast cells for inclusion in a vaccine composition according to a particular embodiment based on various considerations, including the nature of the immunogen included in the recombinant yeast cell, the copy number of the immunogen included in the recombinant yeast cell, and other considerations. Examples of suitable numbers of recombinant yeast cells for inclusion in a vaccine composition according to an embodiment of the invention include, but are not limited to, one, at least one, more than one, two, a plurality, three, four, five, six, seven, eight, nine, ten, more than ten, one hundred, at least one hundred, more than one hundred, one thousand, at least one thousand, more than one thousand, one million, at least one million, and more than one million. Examples of suitable ranges of numbers of recombinant yeast cells for inclusion in a vaccine composition according to an embodiment of the invention include, but are not limited to, between about 1 and about 10⁷, between about 1 and about 10⁶, between about 1 and about 10⁵, between about 1 and about 10⁴, between about 1 and about 10³, between about 1 and about 10², and between about 1 and about 10.

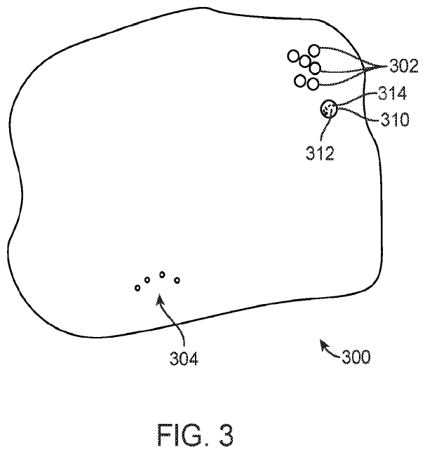

FIG. 3 illustrates a food composition 300 that comprises a vaccine composition 302 according to an embodiment of the invention and at least one foodstuff 304. The vaccine composition 302 can comprise any vaccine composition according to an embodiment. Thus, the vaccine composition comprises an ingestible vessel 310 defining a cavity 312 and at least one recombinant yeast cell 314 according to an embodiment disposed in the cavity 312. In the food composition 300 illustrated in FIG. 3, the ingestible vessel 310 comprises a polymeric shell that has been sprayed onto a plurality of recombinant yeast cells 314 to microencapsulate the recombinant yeast cells 314 in the cavity 312 defined by the ingestible vessel 310 formed by the polymeric shell.

The food composition 300 includes at least one vaccine composition 302 and, as illustrated in FIG. 3, more than one vaccine composition can be included. Indeed, any suitable number of vaccine compositions can be included in a food composition according to a particular embodiment, and a skilled artisan will be able to select a suitable number of vaccine compositions for inclusion in a food composition according to a particular embodiment based on various considerations, including the size, shape, and configuration of the food composition, the nature of the vaccine composition, including the number of recombinant yeast cells included in each vaccine composition included in the food composition, and other considerations. Examples of suitable numbers of vaccine compositions that can be included in a food composition according to an embodiment of the invention include, but are not limited to, one, at least one, more than one, two, a plurality, three, four, five, six, seven, eight, nine, ten, more than ten, between about 1 and about 10⁷, between about 1 and about 10⁶, between about 1 and about 10⁵, between about 1 and about 10⁴, between about 1 and about 10³, between about 1 and about 10², and between about 1 and about 10.

The at least one foodstuff 304 can comprise any substance considered suitable for consumption as food by an animal. Examples include flour, wheat, sugar, butter, bread, dough, meat, yogurt, a fruit or portion thereof, a vegetable or portion thereof, water or another liquid, and combinations of these examples.

The food composition can take any suitable form, including, but not limited to, a cookie, a candy, a bar, a cracker, a wafer, a loaf, a beverage, a yogurt, and any other form considered desirable.

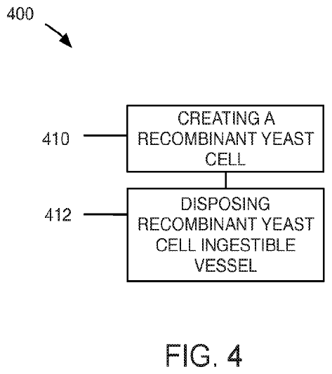

FIG. 4 is a schematic representation of an example method of producing a vaccine 400. An initial step 410 comprises creating a recombinant yeast cell by introducing into a wild-type yeast cell a first nucleic acid sequence encoding a regulated promoter, a second nucleic acid sequence encoding an immunogen, and a third nucleic acid sequence encoding a cell wall permeabilizing agent (e.g., cell wall degrading enzyme). The recombinant yeast cell can comprise any recombinant yeast cell according to an embodiment. Thus at least one, or each, of the first nucleic acid sequence, second nucleic acid sequence, and third nucleic acid sequences can comprise a nucleic acid sequence that does not occur naturally in the wild-type yeast cell from which the recombinant yeast cell is derived. In some embodiments, expression of the second and third nucleic acid sequences is under common genetic control of the regulated promoter. The introducing step 410 can be performed in accordance with any suitable technique or method, including conventional transformation techniques and methods.

Another step 412 comprises disposing the recombinant yeast cell in a cavity defined by an ingestible vessel to produce a vaccine composition in accordance with an embodiment.

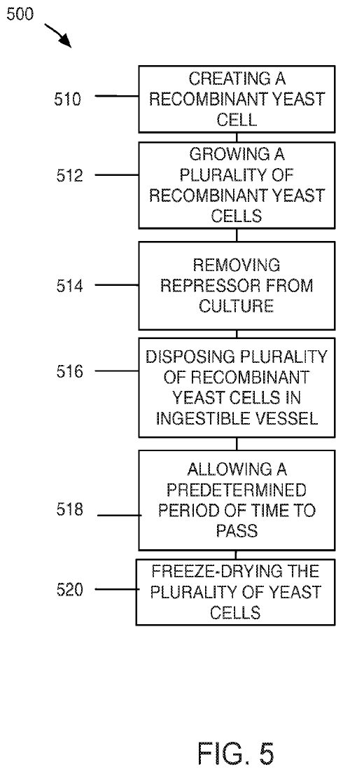

FIG. 5 is a schematic representation of another example method of producing a vaccine 500. An initial step 510 comprises creating a recombinant yeast cell by introducing into a wild-type yeast cell a first nucleic acid sequence encoding a positive repressible promoter that is repressed in the presence of a repressor, a second nucleic acid sequence encoding an immunogen, and a third nucleic acid sequence encoding a cell wall permeabilizing agent (e.g., cell wall degrading enzyme). The recombinant yeast cell can comprise any recombinant yeast cell according to an embodiment. Thus at least one, or each, of the first nucleic acid sequence, second nucleic acid sequence, and third nucleic acid sequences can comprise a nucleic acid sequence that does not occur naturally in the wild-type yeast cell from which the recombinant yeast cell is derived. Also, in some cases, expression of the second and third nucleic acid sequences is under common genetic control of the regulated promoter. The introducing step 510 can be performed in accordance with any suitable technique or method, including conventional transformation techniques and methods.

The positive repressible promoter can comprise any suitable positive repressible promoter. As described above, the inventors have determined that the Tet-off promoter is considered advantageous. In these embodiments, the repressor comprises tetracycline or a tetracycline derivative.

Another step 512 comprises growing a plurality of recombinant yeast cells derived from the recombinant yeast cell in a culture comprising the repressor.

Another step 514 comprises removing the repressor from the culture.

Another step 516 comprises disposing the plurality of recombinant yeast cells in a cavity defined by an ingestible vessel;

An optional step 518 comprises allowing a pre-defined period of time to pass between the step 514 of removing the repressor from the culture and the step 516 of disposing the plurality of recombinant yeast cells in a cavity defined by an ingestible vessel. Inclusion of this optional step 518 is considered advantageous at least because it enables activation of the promoter and, as a result, expression of the second and third nucleic acid sequences for a period of time before disposing the plurality of recombinant yeast cells in a cavity defined by an ingestible vessel.

Another optional step 520 comprises freeze-drying the plurality of recombinant yeast cells. If included, this step 520 can be performed before, concurrently with, or after the step 516 of disposing the plurality of recombinant yeast cells in a cavity defined by an ingestible vessel.

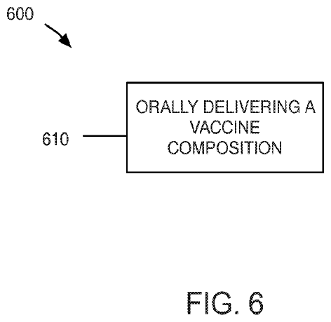

FIG. 6 is a schematic representation of an example method of vaccinating an animal 600. A step 610 comprises orally delivering to the animal to be vaccinated a vaccine composition according to an embodiment.

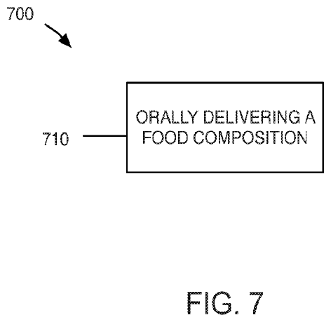

FIG. 7 is a schematic representation of another example method of vaccinating an animal 700. A step 710 comprises orally delivering to the animal to be vaccinated a food composition according to an embodiment.

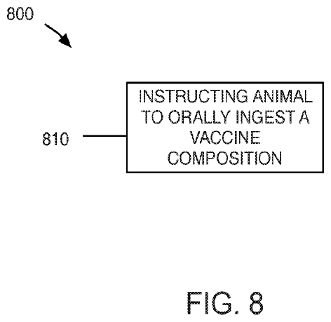

FIG. 8 is a schematic representation of another example method of vaccinating an animal 800. A step 810 comprises instructing the animal to be vaccinated to orally ingest a vaccine composition according to an embodiment.

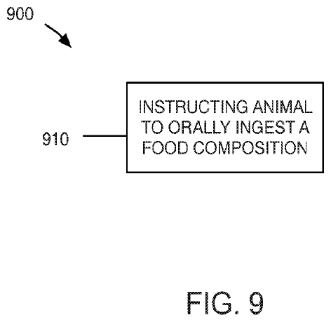

FIG. 9 is a schematic representation of another example method of vaccinating an animal 900. A step 910 comprises instructing the animal to be vaccinated to orally ingest a food composition according to an embodiment.

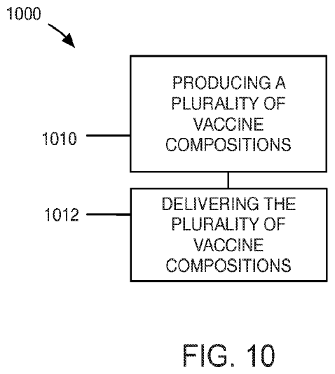

FIG. 10 is a schematic representation of an example method of supplying a vaccine 1000. An initial step 1010 comprises producing a plurality of vaccine compositions, each vaccine composition of the plurality of vaccine compositions comprising a vaccine composition according to an embodiment. A later step 1012 comprises delivering the plurality of vaccine compositions to an individual designated for delivering individual vaccine compositions of the plurality of vaccine compositions to individual animals of said plurality of animals for the purpose of vaccinating individual animals of said plurality of animals.

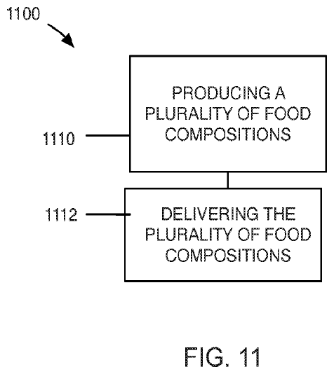

FIG. 11 is a schematic representation of an example method of supplying a vaccine 1100. An initial step 1110 comprises producing a plurality food compositions, each food composition of the plurality of food compositions comprising a food composition according to an embodiment. A later step 1112 comprises delivering the plurality of food compositions to an individual designated for delivering individual food compositions of the plurality of food compositions to individual animals of said plurality of animals for the purpose of vaccinating individual animals of said plurality of animals.

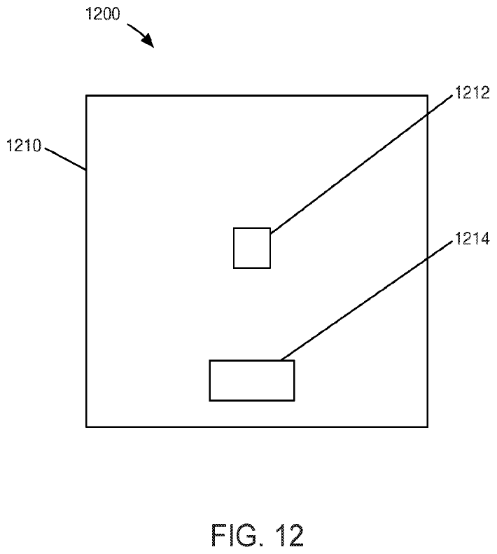

FIG. 12 is a schematic representation of an example kit 1200. Kit 1200 includes a packaging substrate 1210, such as a container or a sheet of material, a vaccine composition 1212 according to an embodiment disposed on or in the packaging substrate 1210, as appropriate, and instructions 1214. The instructions 1314 can include instructions for orally delivering the vaccine composition to an animal, instructions for orally ingesting the vaccine composition, or both.

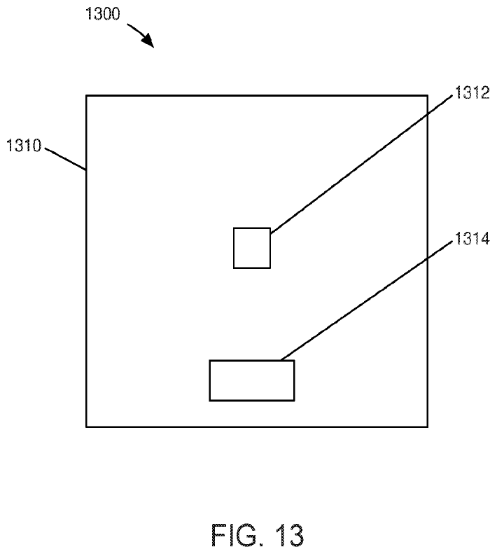

FIG. 13 is a schematic representation of an example kit 1300. Kit 1300 includes a packaging substrate 1310, such as a container or a sheet of material, a food composition 1312 according to an embodiment disposed on or in the packaging substrate 1310, as appropriate, and instructions 1314. The instructions 1314 can include instructions for orally delivering the food composition to an animal, instructions for orally ingesting the food composition, or both.

### EXAMPLES

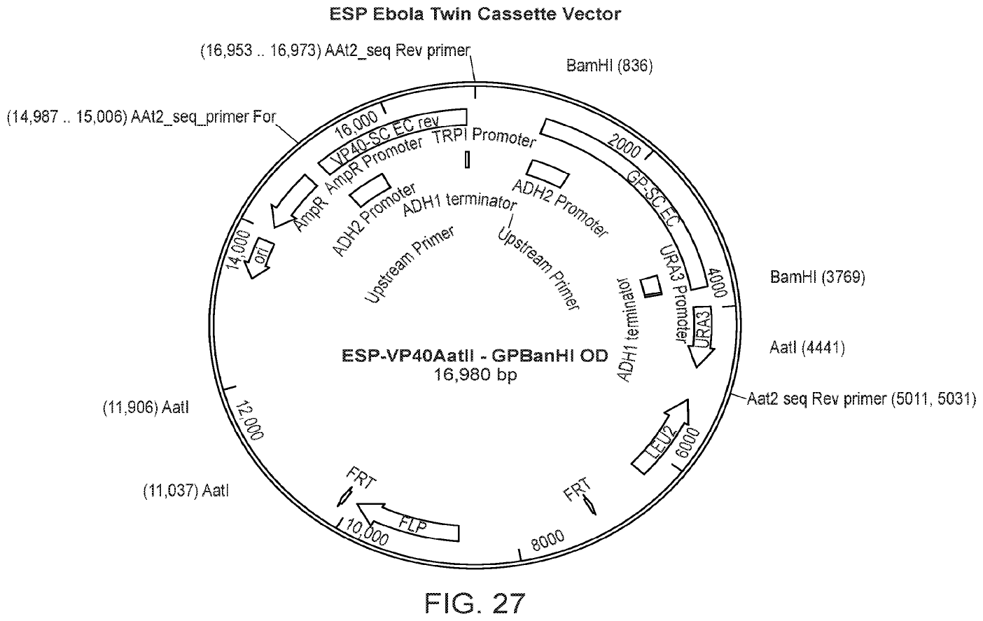

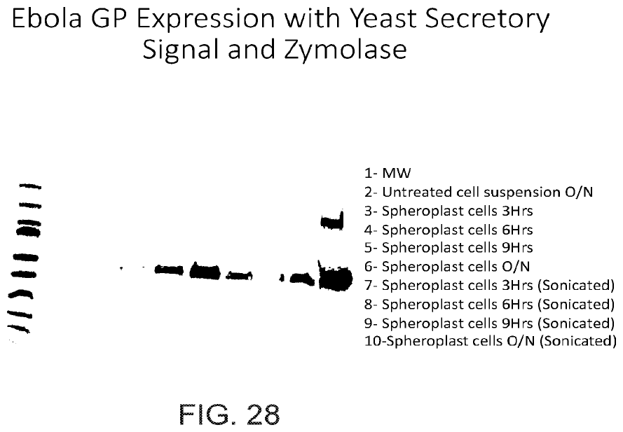

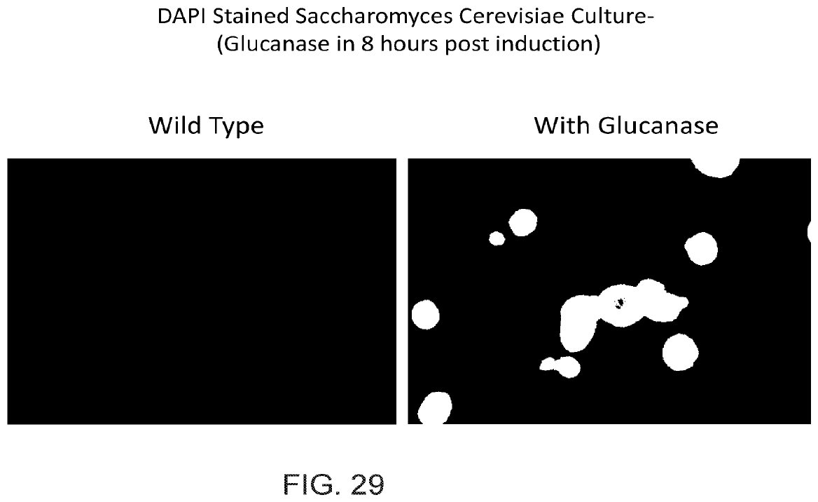

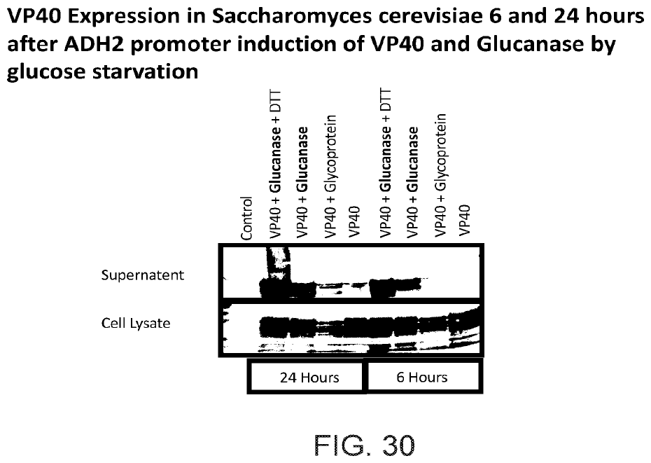

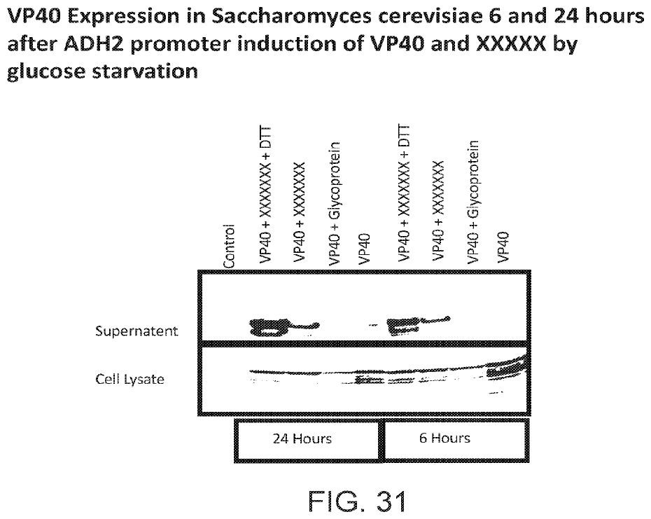

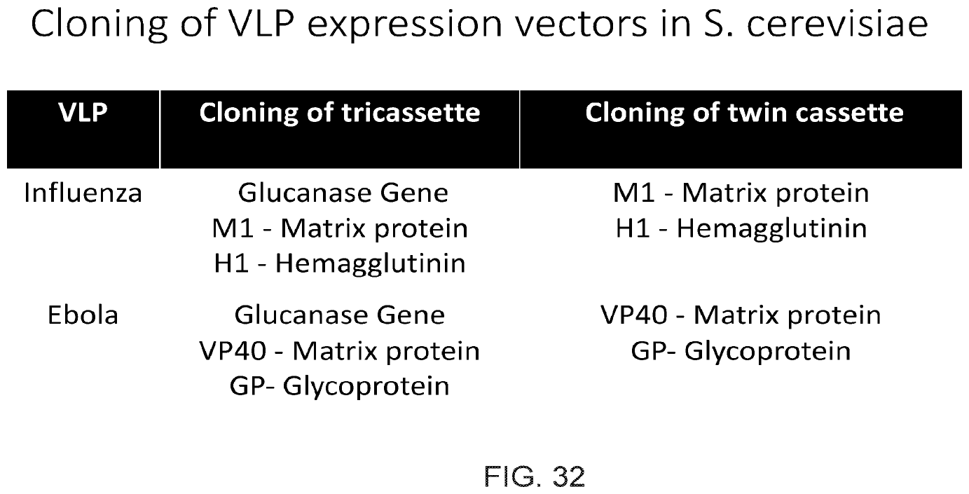

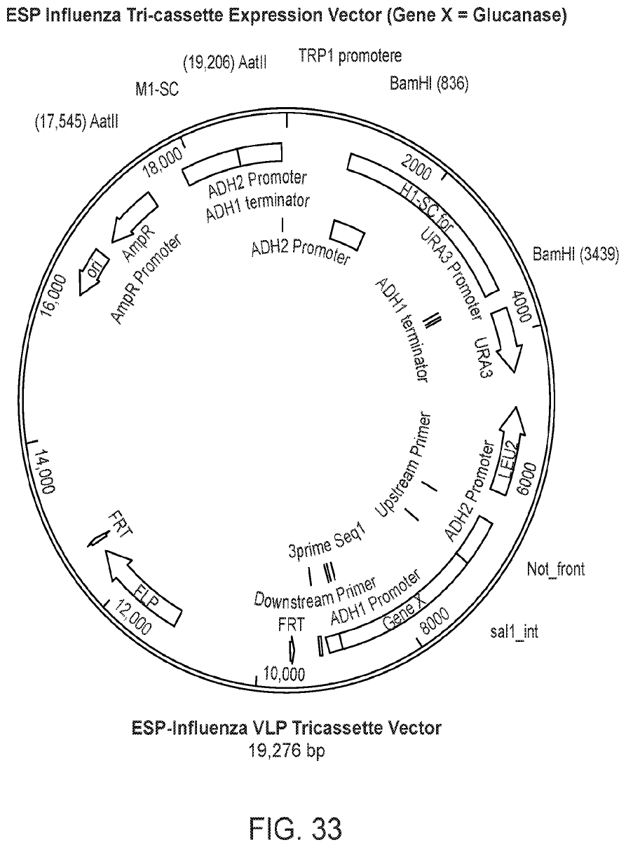

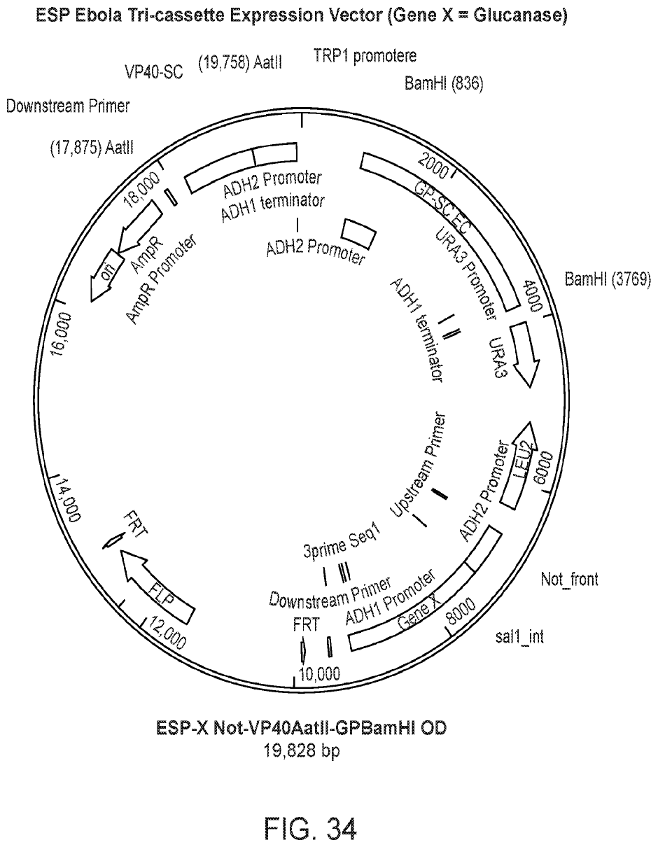

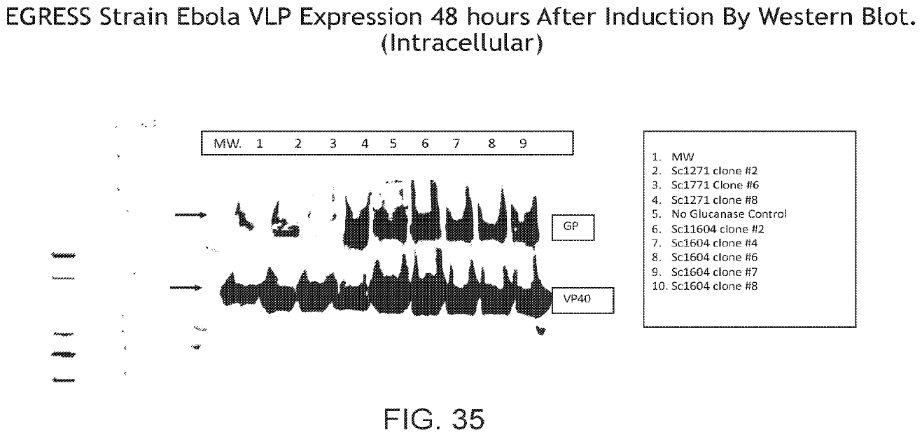

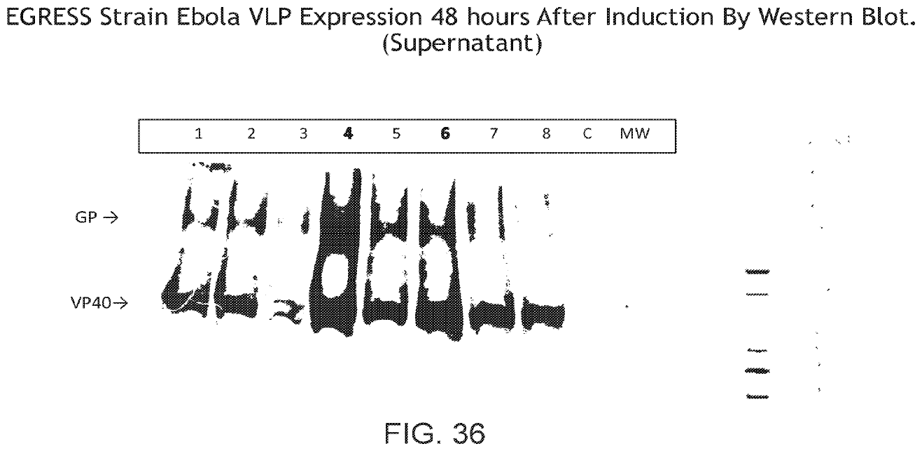

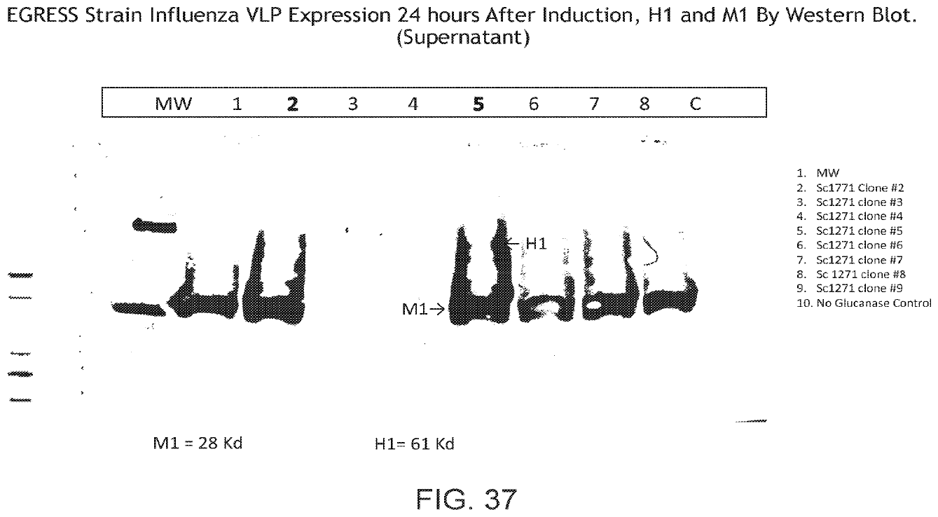

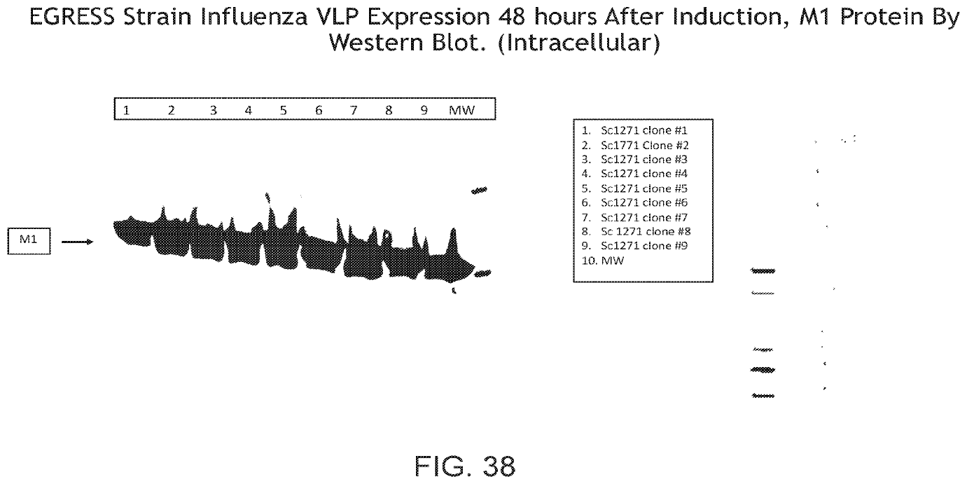

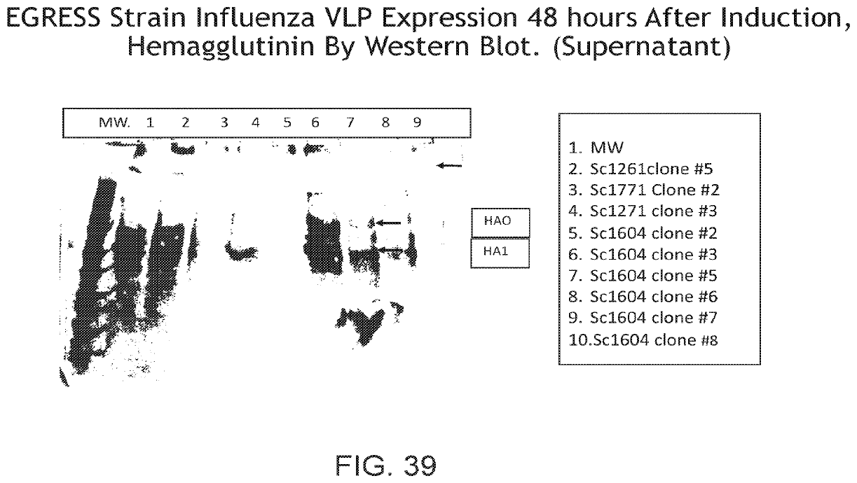

Various examples are illustrated in FIGS. 27-40.

### Example 1: H1/N1 eVLP Production

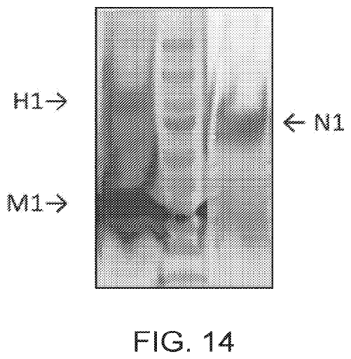

Hansenula polymorpha cells were transformed with nucleic acid encoding human influenza H1, N1, and M1 proteins a recombinant beta-glucanase each under the control of an inducible promoter. Recombinant cells were cultured in shake flasks to approximately OD₆₀₀=10 and induced. Recombinant cells were cultured under inducing conditions and after sufficient time, the culture medium and cells were separated by centrifugation. A sample of cell-depleted culture media was harvested, heated in SDS-PAGE sample buffer to denature proteins, and fractionated by SDS-PAGE. The fractionated proteins were transferred to a blotting membrane and probed for the presence of H1, N1, and M1 proteins. The results showed high levels of H1, N1, and M1 proteins in the culture media, indicating strong secretion of eVLPs in the culture media. FIG. 14.

### Example 2: HIV GAG-MS2 eVLP Production

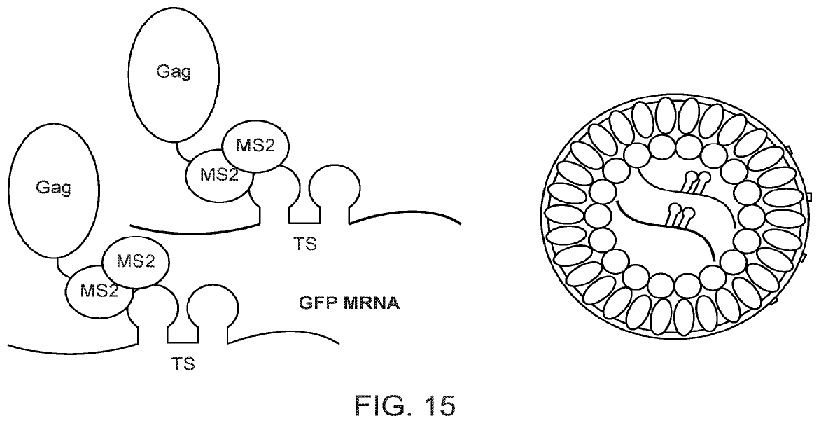

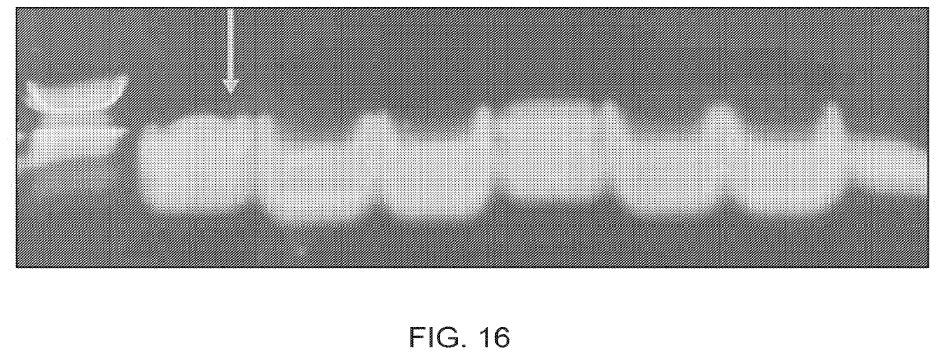

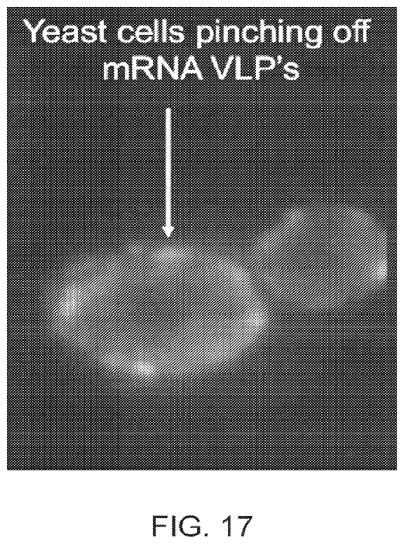

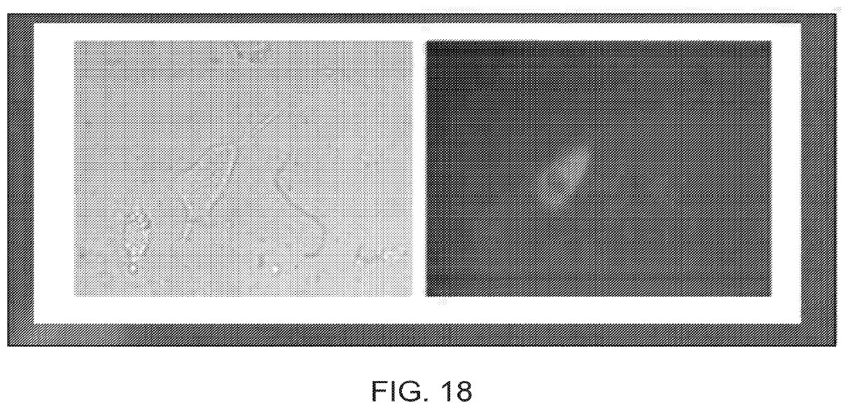

FIG. 15 illustrates a schematic diagram of eVLP encoded by the constructs produced in this example. Yeast cells were transformed with nucleic acid encoding HIV-GAG fusion protein and EGFP-MS2 mRNA, cultured in shake flasks to approximately OD₆₀₀=10 and induced. Recombinant cells were cultured under inducing conditions and yeast cells were harvested from the culture medium after induction. Culture medium was also harvested to obtain eVLP. RT-PCR analysis confirmed the presence of EGFP mRNA in the eVLP (FIG. 16). Microscopy of recovered yeast cells demonstrates secretion of eVLPs (FIG. 17). eVLPs were purified and incubated with dendritic cells (DCs). After 24 hours incubation with DCs, fluorescence microscopy confirmed EGFP production in the DCs (FIG. 18). These results indicate that the DCs phagocytosed the eVLPs and translated the EGFP mRNA for expression of functional EGFP.

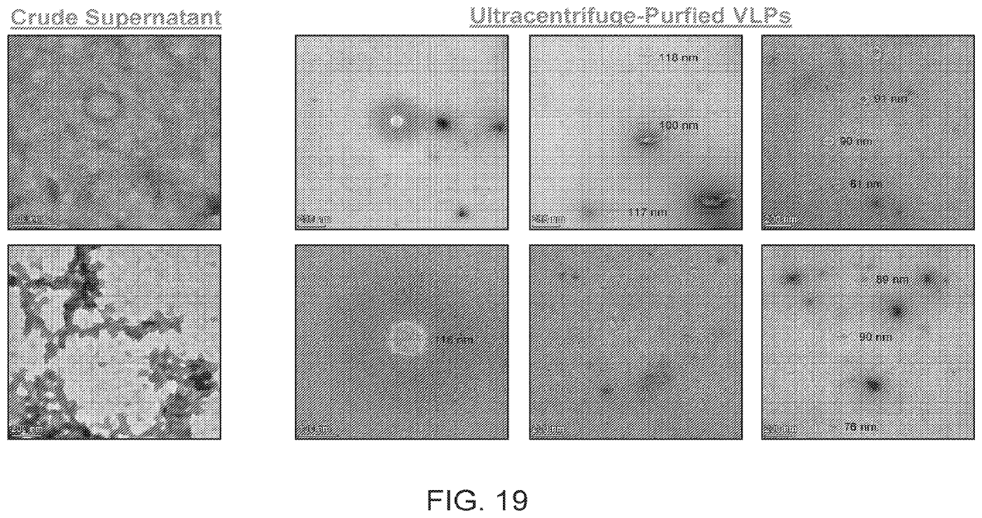

eVLPs were also analyzed by transmission electron microscopy as crude culture supernatant (FIG. 19, left) and after ultracentrifugation to obtain purified eVLPs (FIG. 19, right). The purified eVLPs were approximately 80 to 120 nm in diameter with an envelope thickness of about 4.2 nm.

### Example 3: HIV GAG-GFP eVLP Formulation and Oral Administration

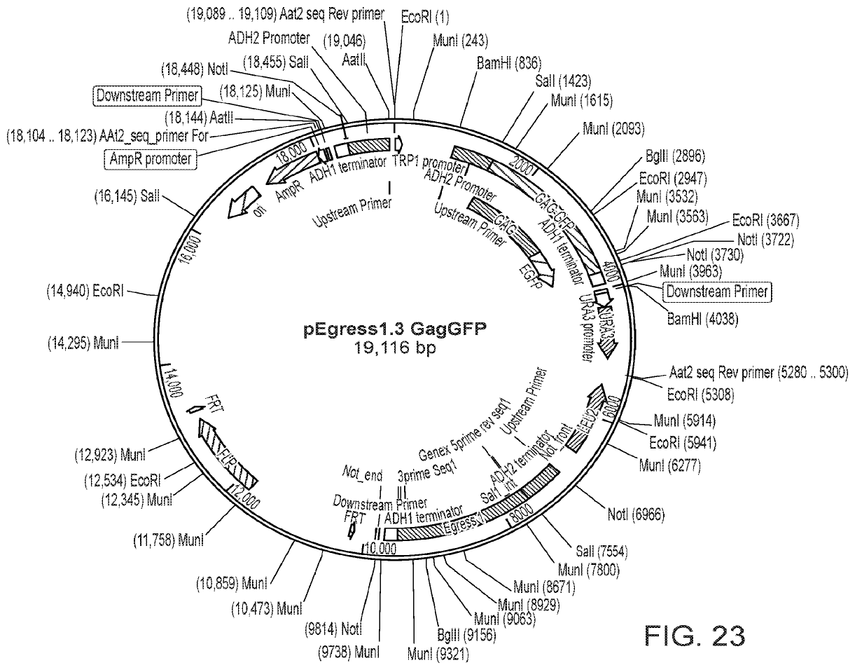

Recombinant yeast cells were transformed with a construct as illustrated in FIG. 23. Recombinant cells were cultured in shake flasks to approximately OD₆₀₀=10, induced, and cultured under inducing conditions. The culture medium was then harvested for formulation.

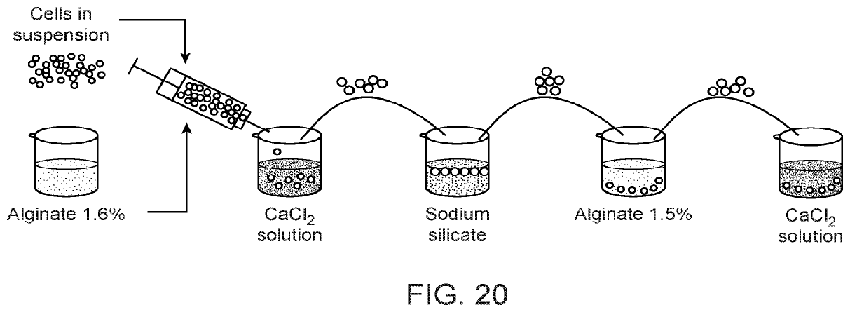

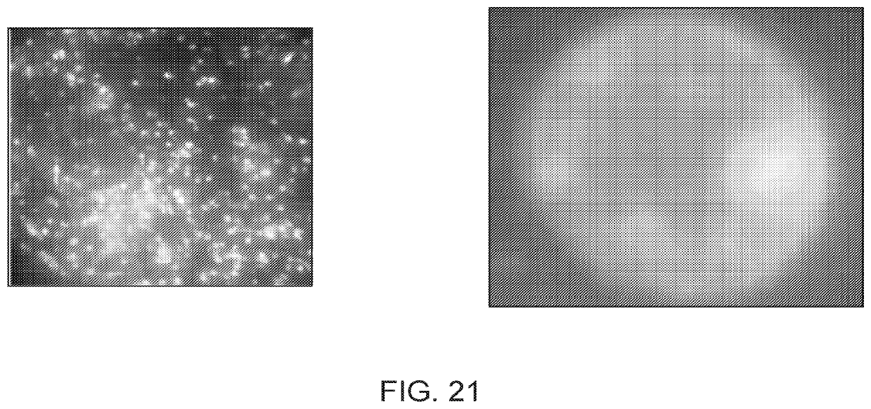

FIG. 20 illustrates a schematic diagram of the formulation strategy. Briefly, harvested cells were suspended in a 1.6% alginate solution and mixed with a CaCl₂) solution to form microencapsulated eVLP producing yeast cells. The microencapsulated material was then transferred to a sodium silicate solution, then a 1.5% alginate solution, and finally a CaCl₂) solution. The resulting material produced enteric-coated microspheres of approximately 100 μm in diameter (FIG. 21).

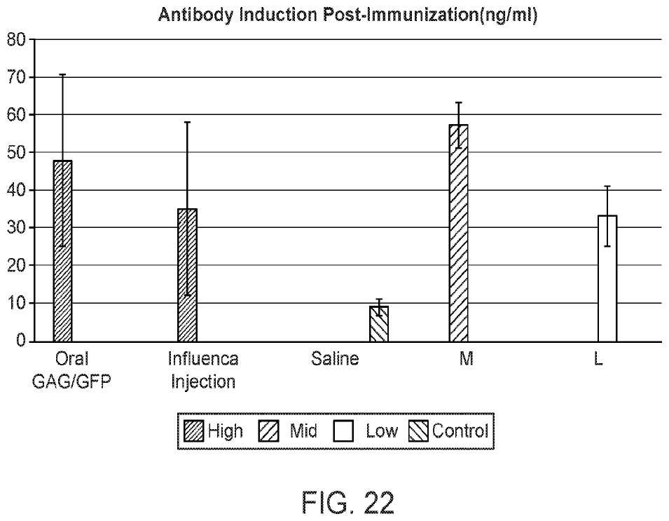

The microsphere-encapsulated yeast cells were administered orally to BABL/c mice in four cohorts (high dose, medium dose, low dose, and saline control). The results demonstrated detectable GFP expression in mice administered the high, medium, and low doses, but not in saline administered mice. Anti-GFP serum antibodies were detected. Induced anti-GFP antibody concentrations were similar to anti-H1 serum levels obtained 20 days after administration of standard H1 injectable vaccine (FIG. 22).

The foregoing detailed description refers to various example recombinant yeast suitable for use in oral vaccination, vaccine compositions, methods of vaccinating an animal, and related methods, kits, and nucleic acid molecules. The description and appended drawings illustrating these various examples are intended only to provide examples of the subject matter which the inventors believe to be within the scope of their invention and are not intended to limit the scope of any claim in any manner. All publications, patents, patent applications, and patent publications disclosed herein are hereby incorporated by reference in the entirety and for all purposes and to the same extent as if each disclosed publication, patent, patent application, or patent publication were specifically incorporated by reference.

Embodiments described herein include:

1. A recombinant yeast cell derived from a wild-type yeast cell, said recombinant yeast cell comprising:

a first nucleic acid sequence encoding a regulated promoter;

a second nucleic acid sequence encoding a VLP immunogen; and

a third nucleic acid sequence encoding a cell wall degrading enzyme;

preferably wherein each of the first nucleic acid sequence, second nucleic acid sequence, and third nucleic acid sequences comprises a nucleic acid sequence that does not occur naturally in said wild-type yeast cell; and

preferably wherein expression of the second and third nucleic acid sequences is under common genetic control of the regulated promoter.

3. The recombinant yeast cell of embodiment 1, wherein the second nucleic acid sequence is positioned downstream of, and under the genetic control of, a first copy of the first nucleic acid sequence and the third nucleic acid sequence is positioned downstream of, and under the genetic control of, a second copy of the first nucleic acid sequence.
4. The recombinant yeast cell of embodiment 3, wherein the first copy of the first nucleic acid sequence and the second nucleic acid sequence are positioned on the same nucleic acid molecule as the second copy of the first nucleic acid sequence and the third nucleic acid sequence.
5. The recombinant yeast cell of embodiment 3, wherein the first copy of the first nucleic acid sequence and the second nucleic acid sequence are positioned on a first nucleic acid molecule and the second copy of the first nucleic acid sequence and the third nucleic acid sequence are positioned on a second nucleic acid molecule; or
The recombinant yeast cell of any one of the foregoing embodiments, wherein the yeast cell comprises the cell wall degrading enzyme (e.g., glucanase), a VLP matrix protein, and an immunogen, optionally wherein the cell wall degrading enzyme, and/or the VLP matrix protein, and/or each of the cell wall degrading enzyme, VLP matrix protein, and immunogen are under the control of a regulatable promoter, and optionally wherein one or more, or each, of the regulatable promoter(s) is the same promoter, a different copy of the same promoter, or a different promoter.
6. The recombinant yeast cell of embodiment 1, wherein the regulated promoter comprises an inducible promoter, preferably wherein the regulated promoter comprises a repressible promoter; more preferably wherein the regulated promoter comprises a positive repressible promoter.
7. The recombinant yeast cell of embodiment 2, wherein the regulated promoter comprises the Tet-off regulated promoter.
8. The recombinant yeast cell of embodiment 1, wherein the VLP immunogen comprises one or more proteins from an influenza virus, preferably wherein the VLP immunogen comprises an M1 matrix protein, a hemagglutinin protein, and a neuraminidase protein from an influenza virus.
9. The recombinant yeast cell of embodiment 8, wherein the VLP immunogen comprises Human Flu M1 matrix, hemagglutinin, and neuraminidase proteins from an influenza virus.
10. The recombinant yeast cell of embodiment 1, wherein the cell wall degrading enzyme comprises a glucanase, preferably wherein the cell wall degrading enzyme comprises a β-glucanase.
11. The recombinant yeast cell of embodiment 10, wherein the cell wall degrading enzyme comprises β-1,3-glucanase.
12. The recombinant yeast cell of embodiment 1, wherein the cell wall degrading enzyme comprises a mannanase.
13. The recombinant yeast cell of embodiment 1, further comprising a fourth nucleic acid sequence that encodes a cell wall inhibiting toxin;

wherein the fourth nucleic acid sequence does not occur naturally in said wild-type yeast cell; preferably wherein expression of the second, third, and fourth nucleic acid sequences are under common genetic control of the regulated promoter.

14. The recombinant yeast cell of any preceding embodiment, wherein said wild-type yeast cell comprises Saccharomyces cerevisiae.
15. The recombinant yeast cell of any preceding embodiment, wherein said recombinant yeast cell is freeze-dried.
16 A recombinant yeast cell derived from a wild-type yeast cell, said recombinant yeast cell comprising:

a first nucleic acid sequence encoding a regulated promoter;

a second nucleic acid sequence encoding one or more proteins from an influenza virus; and

a third nucleic acid sequence encoding a glucanase;

preferably wherein each of the first nucleic acid sequence, second nucleic acid sequence, and third nucleic acid sequences comprising a nucleic acid sequence that does not occur naturally in said wild-type yeast cell;

preferably wherein expression of the second and third nucleic acid sequences is under common genetic control of the regulated promoter.

17. A recombinant yeast cell derived from a wild-type Saccharomyces cerevisiae yeast cell, said recombinant yeast cell comprising:

a first nucleic acid sequence encoding the Tet-off regulated promoter;

a second nucleic acid sequence encoding Human Flu M1 matrix, hemagglutinin, and neuraminidase proteins from an influenza virus; and

a third nucleic acid sequence encoding a glucanase;

preferably wherein each of the first nucleic acid sequence, second nucleic acid sequence, and third nucleic acid sequences comprising a nucleic acid sequence that does not occur naturally in said wild-type yeast cell; and

preferably wherein expression of the second and third nucleic acid sequences is under common genetic control of the regulated promoter.

18. A recombinant yeast cell derived from a wild-type yeast cell, said recombinant yeast cell comprising:

a first nucleic acid sequence encoding a regulated promoter;

a second nucleic acid sequence encoding a VLP immunogen; and

a third nucleic acid sequence encoding a cell wall inhibiting toxin;

preferably wherein each of the first nucleic acid sequence, second nucleic acid sequence, and third nucleic acid sequences comprising a nucleic acid sequence that does not occur naturally in said wild-type yeast cell; and

preferably wherein expression of the second and third nucleic acid sequences is under common genetic control of the regulated promoter.

19. A freeze-dried recombinant yeast cell derived from a wild-type yeast cell and transformed to include a nucleic acid sequence encoding a VLP immunogen and a nucleic acid sequence encoding a cell wall degrading enzyme that are under common genetic control of a regulated promoter that does not occur naturally in said wild-type yeast cell.
20. A vaccine composition, comprising:

an ingestible vessel defining a cavity; and

a recombinant yeast cell derived from a wild-type yeast cell and disposed in the cavity, the recombinant yeast cell comprising a first nucleic acid sequence encoding a regulated promoter, a second nucleic acid sequence encoding a VLP immunogen, and a third nucleic acid sequence encoding a cell wall degrading enzyme;

wherein expression of the second and third nucleic acid sequences is under common genetic control of the regulated promoter.

21. The vaccine composition of embodiment 20, wherein the ingestible vessel comprises a capsule.
22. The vaccine composition of embodiment 21, wherein the capsule is acid-resistant, and delivers substantially functional yeast and/or VLP to an intestine of a subject after oral administration.
23. The vaccine composition of embodiment 21, wherein the capsule defines one or more pores, e.g., pores in a solid capsule that can be induced to release cells and/or VLP, such as mechanically inducible pores.
24. The vaccine composition of embodiment 20, wherein the recombinant yeast cell comprises a recombinant yeast cell according to any of embodiment 1 through 19.
25. A vaccine composition, comprising:

an ingestible capsule defining a cavity; and

a freeze-dried recombinant yeast cell derived from a wild-type yeast cell and disposed in the cavity, the recombinant yeast cell comprising a first nucleic acid sequence encoding a regulated promoter, a second nucleic acid sequence encoding a VLP immunogen, and a third nucleic acid sequence encoding a cell wall degrading enzyme;

preferably wherein each of the first nucleic acid sequence, second nucleic acid sequence, and third nucleic acid sequences comprising a nucleic acid sequence that does not occur naturally in said wild-type yeast cell; and

preferably wherein expression of the second and third nucleic acid sequences is under common genetic control of the regulated promoter.

26. A food composition, comprising:

at least one foodstuff; and

at least one vaccine composition, comprising
an ingestible vessel defining a cavity; and
a recombinant yeast cell disposed in the cavity, or a plurality of recombinant yeast cells, wherein said recombinant yeast cell comprises a first nucleic acid sequence encoding a regulated promoter, a second nucleic acid sequence encoding a VLP immunogen, and a third nucleic acid sequence encoding a cell wall degrading enzyme;
each of the first nucleic acid sequence, second nucleic acid sequence, and third nucleic acid sequences comprising a nucleic acid sequence that does not occur naturally in said wild-type yeast cell; and
expression of the second and third nucleic acid sequences is under common genetic control of the regulated promoter.
   27. The food composition of embodiment 26, wherein the ingestible vessel comprises a polymeric shell that has been sprayed onto the recombinant yeast cell, or onto the plurality of yeast cells.
   28. The food composition of embodiment 27, wherein the at least one vaccine composition comprises more than one vaccine composition.
   29. The food composition of embodiment 26, wherein the ingestible vessel of each vaccine composition of the more than one vaccine composition comprises a polymeric shell that has been sprayed onto the recombinant yeast cell.
   30. The food composition of embodiment 26, wherein the foodstuff comprises one or more of flour, wheat, sugar, butter, bread, dough, meat, a fruit or portion thereof, and a vegetable or portion thereof.
   31. The food composition of embodiment 26, wherein said food composition is in the form of a cookie, a candy, a bar, a cracker, a wafer, a loaf, or a drink.
   32. A food composition, comprising:

at least one foodstuff; and

a plurality of vaccine compositions, each vaccine composition of the plurality of vaccine compositions comprising
a polymeric shell defining a cavity; and
a plurality of recombinant yeast cells disposed in the cavity, each recombinant yeast cell of the plurality of recombinant yeast cells comprising a first nucleic acid sequence encoding a regulated promoter, a second nucleic acid sequence encoding a VLP immunogen, and a third nucleic acid sequence encoding a cell wall degrading enzyme;
each of the first nucleic acid sequence, second nucleic acid sequence, and third nucleic acid sequences comprising a nucleic acid sequence that does not occur naturally in said wild-type yeast cell; and
expression of the second and third nucleic acid sequences is under common genetic control of the regulated promoter.
   33. A food composition, comprising at least one foodstuff and a vaccine composition comprising a plurality of recombinant yeast cells disposed in a cavity defined by a polymeric shell, each recombinant yeast cell of the plurality of recombinant yeast cells is derived from a wild-type yeast cell and transformed to include a nucleic acid sequence encoding a VLP immunogen and a nucleic acid sequence encoding a cell wall degrading enzyme that are under common genetic control of a regulated promoter that does not occur naturally in said wild-type yeast cell.
   34. The food composition of embodiment 33, wherein each recombinant yeast cell of the plurality of recombinant yeast cells is freeze-dried.
   35. A method of vaccinating an animal, said method comprising orally delivering to said animal a vaccine composition according to an embodiment.
   36. A method of vaccinating an animal, said method comprising orally delivering to said animal a food composition according to an embodiment.
   37. A kit, comprising:
   a packaging substrate;

the vaccine composition according to embodiment 20;

and instructions for using the vaccine composition.

38. The kit of embodiment 37, wherein the packaging substrate comprises a sheet of material.
39. The kit of embodiment 38, wherein the vaccine composition is disposed on the packaging substrate.
40. The kit of embodiment 39, wherein the vaccine composition is secured to the packaging substrate.
41. The kit of embodiment 40, further comprising shrink wrap material securing the vaccine composition to the packaging substrate.
42. The kit of embodiment 37, wherein the packaging substrate comprises a container.
43. The kit of embodiment 37, wherein the instructions for using the vaccine composition comprises instructions for orally administering the vaccine composition to an animal.
44. The kit of embodiment 37, wherein the instructions for using the vaccine composition comprise instructions for orally ingesting the vaccine composition.
45. A kit, comprising:

a packaging substrate;

the food composition according to embodiment 26;

and instructions for using the food composition.

46. The kit of embodiment 45, wherein the packaging substrate comprises a sheet of material.
47. The kit of embodiment 46, wherein the food composition is disposed on the packaging substrate.
48. The kit of embodiment 47, wherein the food composition is secured to the packaging substrate.
49. The kit of embodiment 48, further comprising shrink wrap material securing the food composition to the packaging substrate.
50. The kit of embodiment 45, wherein the packaging substrate comprises a container.
51. The kit of embodiment 45, wherein the instructions for using the food composition comprises instructions for orally administering the food composition to an animal.
52. The kit of embodiment 45, wherein the instructions for using the food composition comprise instructions for orally ingesting the food composition.
53. A recombinant yeast cell derived from a wild-type yeast cell, said recombinant yeast cell comprising:

a first nucleic acid sequence encoding a regulated promoter;

a second nucleic acid sequence encoding a Gag-MS2 fusion protein;

a third nucleic acid sequence encoding a cell wall degrading enzyme; and

a fourth nucleic acid sequence encoding a non-translated MS2 binding sequence and an antigen;

preferably wherein each of the first nucleic acid sequence, second nucleic acid sequence, third nucleic acid sequence, and the fourth nucleic acid sequence comprises a nucleic acid sequence that does not occur naturally in said wild-type yeast cell; and

preferably wherein expression of the second and third nucleic acid sequences is under common genetic control of the regulated promoter.

54. The recombinant yeast cell of embodiment 53, wherein expression of the second, third, and fourth nucleic acid sequences is under common genetic control of the regulated promoter.
55. The recombinant yeast cell of embodiment 53, wherein the fourth nucleic acid sequence is derived from one of influenza virus, RSV, and HIV.
56. A recombinant yeast cell derived from a wild-type yeast cell, said recombinant yeast cell comprising:

a first nucleic acid sequence encoding a regulated promoter;

a second nucleic acid sequence comprising at least a portion of each of Parainfluenza 5 NP, V/P, and L genes and lacking one or more of the Parainfluenza 5 genes selected from the group consisting of M, F, SH, and HN;

a third nucleic acid sequence encoding a cell wall degrading enzyme; and

a fourth nucleic acid sequence encoding an antigen;

preferably wherein each of the first nucleic acid sequence, second nucleic acid sequence, third nucleic acid sequence, and the fourth nucleic acid sequence comprises a nucleic acid sequence that does not occur naturally in said wild-type yeast cell; and

preferably wherein expression of the second and third nucleic acid sequences is under common genetic control of the regulated promoter.

57. The recombinant yeast cell of embodiment 56, wherein expression of the second, third, and fourth nucleic acid sequences is under common genetic control of the regulated promoter.
58. The recombinant yeast cell of embodiment 56, wherein the fourth nucleic acid sequence is derived from a virus other than Parainfluenza 5.
59. The recombinant yeast cell of embodiment 58, wherein the fourth nucleic acid sequence is derived from one of influenza virus, RSV, and HIV.

We claim:

1. A recombinant yeast cell comprising:
a heterologous regulatable promoter operably linked to a nucleic acid sequence encoding a cell wall permeabilizing agent; and
a heterologous promoter operably linked to a nucleic acid sequence encoding an immunogen or component thereof, and optionally further comprises
a heterologous promoter operably linked to a nucleic acid sequence encoding a viral matrix protein or functional fragment thereof, viral capsid protein or functional fragment thereof, or viral structural protein or functional fragment thereof.

2. The recombinant yeast cell of claim 1, wherein the immunogen comprises a component of a VLP.

3. The recombinant yeast cell of claim 2, wherein the immunogen comprises a structural component of a VLP.

4. The recombinant yeast cell of claim 3, wherein the immunogen comprises a capsid protein, or functional fragment thereof.

5. The recombinant yeast cell of claim 3, wherein the immunogen comprises a matrix protein, or functional fragment thereof.

6. The recombinant yeast cell of claim 2, wherein the immunogen comprises a component of a VLP fused to a nucleic acid binding peptide, preferably wherein the binding peptide comprises an MS2 peptide sequence.

7. The recombinant yeast cell of claim 6, wherein the recombinant yeast cell further comprises a nucleic acid sequence comprising a region comprising a nucleic acid binding peptide ligand sequence and a region encoding for an antigen, preferably wherein the nucleic acid binding peptide ligand sequence comprises an MS2 ligand sequence.

8. The recombinant yeast cell of claim 2, wherein the immunogen comprises an antigen that embeds in a lipid bilayer of an eVLP.

9. The recombinant yeast cell of claim 1, wherein the recombinant yeast cell comprises a nucleic acid sequence encoding a structural component of a VLP and a nucleic acid sequence encoding an antigen that embeds in a lipid bilayer of an eVLP formed from the structural VLP component.

10. The recombinant yeast cell of any one of claims 1 to 9, wherein the immunogen and cell wall permeabilizing agent are under common genetic control.

11. The recombinant yeast cell of any one of claims 1 to 10, wherein the cell wall permeabilizing agent is a beta-glucanase.

12. The recombinant yeast cell of claim 11, wherein the beta-glucanase is a β-1-3-glucanase.

13. The recombinant yeast cell of claim 12, wherein the β-1-3-glucanase comprises a secreted protein sequence encoded by SEQ ID NO. 1, or wherein the β-1-3-glucanase comprises at least 100 contiguous amino acids of, or all of, SEQ ID NO. 10.

14. The recombinant yeast cell of claim 12, wherein the β-1-3-glucanase is at least 95%, or at least 99%, identical to a secreted protein sequence encoded by SEQ ID NO. 1, or wherein the β-1-3-glucanase is at least 95% identical to at least 100 contiguous amino acids of, or all of, SEQ ID NO. 10.

15. The recombinant yeast cell of claim 12, wherein the β-1-3-glucanase comprises no more than 1, 2, 3, 4, or 5 single amino acid substitutions, deletions, and/or additions relative to the protein encoded by SEQ ID NO. 1 or the polypeptide sequence set forth in SEQ ID NO. 10.

16. The recombinant yeast cell of any one of claims 1 to 10, wherein the cell wall permeabilizing agent is a chitinase.

17. The recombinant yeast cell of claim 16, wherein the chitinase comprises a secreted protein sequence encoded by SEQ ID NO. 4, or wherein the chitinase comprises at least 100 contiguous amino acids of, or all of, SEQ ID NO. 12.

18. The recombinant yeast cell of claim 16, wherein the chitinase is at least 95%, or at least 99%, identical to a secreted protein sequence encoded by SEQ ID NO. 4, or wherein the chitinase is at least 95% identical to at least 100 contiguous amino acids of, or all of, SEQ ID NO. 12.

19. The recombinant yeast cell of claim 16, wherein the chitinase comprises no more than 1, 2, 3, 4, or 5 single amino acid substitutions, deletions, and/or additions relative to the protein sequence encoded by SEQ ID NO. 4 or the polypeptide sequence set forth in SEQ ID NO. 12.

20. The recombinant yeast cell of any one of claims 1 to 10, wherein the cell wall permeabilizing agent is a cell wall inhibiting toxin, e.g., encoded by SEQ ID NO. 5, comprises a protein sequence encoded by SEQ ID NO. 5 or set forth in SEQ ID NO. 23, is at least 95%, or at least 99%, identical to a secreted protein sequence encoded by SEQ ID NO. 5 or set forth in SEQ ID NO. 23, or comprises no more than 1, 2, 3, 4, or 5 single amino acid substitutions, deletions, and/or additions relative to the protein sequence encoded by SEQ ID NO. 5 or set forth in SEQ ID NO. 23.

21. A method for producing a vaccine composition, the method comprising:
culturing a recombinant yeast cell according to any one of claims 1 to 20 in a culture medium under conditions where the regulated promoter represses expression of the operably linked nucleic acid sequence; and
inducing expression of the nucleic acid operably linked to the heterologous regulatable promoter, thereby permeabilizing the recombinant yeast cell.

22. The method of claim 21, wherein the method comprises inducing expression of the nucleic acid operably linked to the heterologous regulatable promoter for at least a portion of the culturing and:
harvesting the permeabilized recombinant yeast cell from the culture medium; or
harvesting the immunogen from the culture medium.

23. The method of any one of claims 21 to 22, wherein the method comprises harvesting the recombinant yeast cell, permeabilized recombinant yeast cell, or immunogen and forming a vaccine composition therefrom.

24. The method of claim 23, wherein the method comprises freeze drying harvested recombinant yeast cell or permeabilized recombinant yeast cell and forming the vaccine composition therefrom.

25. The method of any one of claims 21 to 24, wherein the method comprises admixing a foodstuff with the vaccine composition.

26. A method for making a vaccine composition comprising a recombinant yeast cell, the method comprising:
providing a recombinant yeast cell according to any one of claims 1 to 20; and
admixing the recombinant yeast cell with a pharmaceutically acceptable excipient or foodstuff.

27. A vaccine composition comprising a recombinant yeast cell according to any one of claims 1 to 20 and a pharmaceutically acceptable excipient or foodstuff.

28. A method of administering a vaccine to a subject, the method comprising:
providing a vaccine composition according to claim 27; and
orally administering the vaccine composition.
